(19) United States
(12) Patent Application Publication
(10) Pub. No.: US 2024/0409687 A1
(43) Pub. Date: Dec. 12, 2024

(54) AROMATIC POLYCARBONATE RESIN, POLYCARBONATE RESIN COMPOSITION AND MOLDED ARTICLE

(71) Applicant: IDEMITSU KOSAN CO.,LTD., Tokyo (JP)

(72) Inventor: Daisuke YOSHIZAWA, Funabashi-shi, Chiba (JP)

(73) Assignee: IDEMITSU KOSAN CO.,LTD., Tokyo (JP)

(21) Appl. No.: 18/699,736

(22) PCT Filed: Oct. 28, 2022
(86) PCT No.: PCT/JP2022/040291

§ 371 Date: Apr. 9, 2024

(30) Foreign Application Priority Data
Oct. 29, 2021 (JP) 2021-178061
Mar. 30, 2022 (JP) 2022-057523

(51) Int. Cl. C08G 64/06; C07C 69/757; C08G 64/24; C08G 64/30

(52) U.S. Cl. CPC C08G 64/06; C07C 69/757; C08G 64/24; C08G 64/307; C07C 2601/08; C07C 2601/14

(57) ABSTRACT

The present application relates to an aromatic polycarbonate-based resin, including a repeating unit represented by the following formula (II):

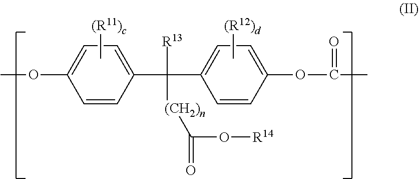

wherein in the formula (II), R¹¹, R¹², R¹³, “c”, “d”, and “n” are as defined above.

### TECHNICAL FIELD

The present invention relates to an aromatic polycarbonate-based resin, a polycarbonate-based resin composition, and a molded article.

### BACKGROUND ART

A polycarbonate-based resin is excellent in, for example, impact resistance, transparency, heat resistance, and self-extinguishing property, and hence has been widely utilized as an engineering plastic in various fields, such as an electrical and electronic equipment field, and an automotive field. However, the polycarbonate-based resin has low surface hardness, and is hence insufficient in scratch resistance in some cases.

In PTL 1, as a polycarbonate copolymer having improved scratch resistance, there is a disclosure of a polycarbonate copolymer including: a unit derived from a hydroxy-terminated monocyclic, polycycic, or fused cyclic compound having a (meth)acrylate group; and a carbonate unit.

In PTL 2, there are disclosures of a fire-resistant polycarbonate resin, which is branched or crosslinked, and an intermediate thereof.

### CITATION LIST

### Patent Literature

PTL 1: KR 2016-0141268 A
PTL 2: JP 02-219818 A

### SUMMARY OF INVENTION

### Technical Problem

A method including coating the surface of the uppermost layer of a structural body formed of a polycarbonate-based resin has been known as a method of improving the surface hardness thereof. However, there is a problem in that a coating step is required, and hence a production process becomes complicated and an environmental load becomes larger.

In addition, a method including blending the structural body with an acrylic resin excellent in surface hardness and transparency such as a polymethyl methacrylate resin has been known. However, the transparency of the structural body tends to be insufficient owing to the occurrence of a phase separation and a difference in refractive index between the resins.

Further, the invention described in PTL 1 is insufficient in surface hardness and transparency. In PTL 2, there is no description of a method of improving the surface hardness of the polycarbonate resin.

As described above, a further investigation has been required to achieve both of transparency and scratch resistance with the polycarbonate-based resin alone.

An object of the present invention is to provide an aromatic polycarbonate-based resin, a polycarbonate-based resin composition, and a molded article each of which is improved in surface hardness without impairment of its appearance, and achieves both of transparency and scratch resistance.

### Solution to Problem

The inventor of the present invention has found that the above-mentioned problems are solved by an aromatic polycarbonate-based resin including a specific repeating unit.

That is, the present invention encompasses the following items 1 to 18.

1. An aromatic polycarbonate-based resin, comprising a repeating unit represented by the following formula (II):

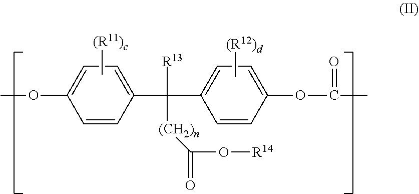

wherein in the formula (II),
R¹¹ and R¹² each independently represent a halogen atom, or a group selected from the group consisting of: an alkyl group having 1 to 18 carbon atoms; an alkoxy group having 1 to 18 carbon atoms; a cycloalkyl group having 3 to 20 carbon atoms; a cycloalkoxy group having 6 to 20 carbon atoms; an alkenyl group having 2 to 10 carbon atoms; an aryl group having 6 to 14 carbon atoms; an aryloxy group having 6 to 14 carbon atoms; an aralkyl group having 7 to 20 carbon atoms; an aralkyloxy group having 7 to 20 carbon atoms; a nitro group; an aldehyde group; a cyano group; and a carboxyl group,
R¹³ represents a hydrogen atom, or a group selected from the group consisting of: an alkyl group having 1 to 5 carbon atoms; a cycloalkyl group having 3 to 20 carbon atoms; a cycloalkoxy group having 3 to 20 carbon atoms; an alkenyl group having 2 to 10 carbon atoms; and an aryl group having 6 to 14 carbon atoms,
R¹⁴ represents a saturated or unsaturated alicyclic group having 3 to 20 carbon atoms, or a 3- to 20-membered saturated or unsaturated heterocyclic group,
“c” and “d” each independently represent an integer of from 0 to 4, and
“n” represents an integer of from 0 to 20.

2. The aromatic polycarbonate-based resin according to the above-mentioned item 1, further comprising a repeating unit represented by the following formula (I), wherein a molar ratio ((I):(II)) between the repeating unit represented by the formula (I) and the repeating unit represented by the formula (II) is from 0:100 to 99.5:0.5:

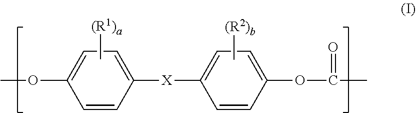

wherein in the formula (I),
R¹ and R² each independently represent a halogen atom, or a group selected from the group consisting of: an alkyl group having 1 to 18 carbon atoms; an alkoxy group having 1 to 18 carbon atoms; a cycloalkyl group having 6 to 20 carbon atoms; a cycloalkoxy group having 6 to 20 carbon atoms; an alkenyl group having 2 to 10 carbon atoms; an aryl group having 6 to 14 carbon atoms; an aryloxy group having 6 to 14 carbon atoms; an aralkyl group having 7 to 20 carbon atoms; an aralkyloxy group having 7 to 20 carbon atoms; a nitro group; an aldehyde group; a cyano group; and a carboxyl group,
X represents a single bond, an alkylene group having 1 to 8 carbon atoms, an alkylidene group having 2 to 8 carbon atoms, a cycloalkylene group having 5 to 15 carbon atoms, a cycloalkylidene group having 5 to 15 carbon atoms, an aralkyl group having 7 to 20 carbon atoms, —S—, —SO—, —SO₂—, —O—, or —CO—, and
“a” and “b” each independently represent an integer of from 0 to 4.

3. The aromatic polycarbonate-based resin according to the above-mentioned item 2, wherein the molar ratio ((I):(II)) between the repeating unit represented by the formula (I) and the repeating unit represented by the formula (II) is from 0.5:99.5 to 99.5:0.5.

4. The aromatic polycarbonate-based resin according to the above-mentioned item 2, wherein the molar ratio ((I):(II)) between the repeating unit represented by the formula (I) and the repeating unit represented by the formula (II) is from 60:40 to 99.5:0.5.

5. The aromatic polycarbonate-based resin according to any one of the above-mentioned items 1 to 4, wherein R¹⁴ represents a saturated or unsaturated alicyclic group having 3 to 12 carbon atoms, or a 3- to 12-membered saturated or unsaturated heterocyclic group.

6. The aromatic polycarbonate-based resin according to any one of the above-mentioned items 1 to 5, wherein R¹⁴ represents a cyclopentyl group or a cyclohexyl group, and “n” represents 2.

7. The aromatic polycarbonate-based resin according to any one of the above-mentioned items 1 to 6, wherein the aromatic polycarbonate-based resin has a viscosity-average molecular weight of from 10,000 to 100,000.

8. The aromatic polycarbonate-based resin according to any one of the above-mentioned items 1 to 7, wherein the aromatic polycarbonate-based resin has a scratch hardness of F or more, which is evaluated in conformity with JIS K5600-5-4.

9. The aromatic polycarbonate-based resin according to any one of the above-mentioned items 1 to 8, wherein the aromatic polycarbonate-based resin has a total light transmittance of 87% or more when molded into a thickness of 1.5 mm.

10. A dihydric phenol-based compound, which is represented by the following formula (ii):

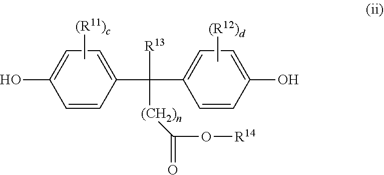

wherein in the formula (ii),
R¹¹ and R¹² each independently represent a halogen atom, or a group selected from the group consisting of: an alkyl group having 1 to 18 carbon atoms; an alkoxy group having 1 to 18 carbon atoms; a cycloalkyl group having 3 to 20 carbon atoms; a cycloalkoxy group having 6 to 20 carbon atoms; an alkenyl group having 2 to 10 carbon atoms; an aryl group having 6 to 14 carbon atoms; an aryloxy group having 6 to 14 carbon atoms; an aralkyl group having 7 to 20 carbon atoms; an aralkyloxy group having 7 to 20 carbon atoms; a nitro group; an aldehyde group; a cyano group; and a carboxyl group,
R¹³ represents a hydrogen atom, or a group selected from the group consisting of: an alkyl group having 1 to 5 carbon atoms; a cycloalkyl group having 3 to 20 carbon atoms; a cycloalkoxy group having 3 to 20 carbon atoms; an alkenyl group having 2 to 10 carbon atoms; and an aryl group having 6 to 14 carbon atoms,
R¹⁴ represents a saturated or unsaturated alicyclic group having 3 to 20 carbon atoms, or a 3- to 20-membered saturated or unsaturated heterocyclic group,
“c” and “d” each independently represent an integer of from 0 to 4, and
“n” represents an integer of from 0 to 20.

11. A method of producing an aromatic polycarbonate-based resin, comprising a step of subjecting a dihydric phenol-based compound and a polycarbonate oligomer to interfacial polycondensation in the presence of a water-insoluble organic solvent and an aqueous solution of an alkali compound, wherein the dihydric phenol-based compound contains a dihydric phenol-based compound (a) represented by the following formula (ii):

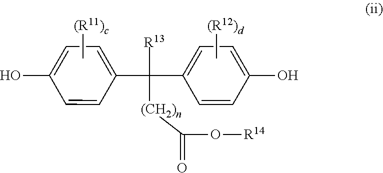

wherein in the formula (ii),
R¹¹ and R¹² each independently represent a halogen atom, or a group selected from the group consisting of: an alkyl group having 1 to 18 carbon atoms; an alkoxy group having 1 to 18 carbon atoms; a cycloalkyl group having 3 to 20 carbon atoms; a cycloalkoxy group having 6 to 20 carbon atoms; an alkenyl group having 2 to 10 carbon atoms; an aryl group having 6 to 14 carbon atoms; an aryloxy group having 6 to 14 carbon atoms; an aralkyl group having 7 to 20 carbon atoms; an aralkyloxy group having 7 to 20 carbon atoms; a nitro group; an aldehyde group; a cyano group; and a carboxyl group,
R¹³ represents a hydrogen atom, or a group selected from the group consisting of: an alkyl group having 1 to 5 carbon atoms; a cycloalkyl group having 3 to 20 carbon atoms; a cycloalkoxy group having 3 to 20 carbon atoms; an alkenyl group having 2 to 10 carbon atoms; and an aryl group having 6 to 14 carbon atoms,
R¹⁴ represents a saturated or unsaturated alicyclic group having 3 to 20 carbon atoms, or a 3- to 20-membered saturated or unsaturated heterocyclic group,
“c” and “d” each independently represent an integer of from 0 to 4, and
“n” represents an integer of from 0 to 20.

12. A polycarbonate-based resin composition, comprising the aromatic polycarbonate-based resin of any one of the above-mentioned items 1 to 9.

13. The polycarbonate-based resin composition according to the above-mentioned item 12, wherein the polycarbonate-based resin composition is for use in a scratch-resistant application.

14. A molded article of the polycarbonate-based resin composition of the above-mentioned item 12 or 13.

15. The molded article according to the above-mentioned item 14, wherein the molded article is a resin window, a touch panel, an interior supply, an exterior supply, an interior part or an exterior part of a vehicle, a casing, an electric appliance, a building material, or OA equipment.

16. A structural body, comprising an outer surface formed of the polycarbonate-based resin composition of the above-mentioned item 12 or 13.

17. A use of the aromatic polycarbonate-based resin of any one of the above-mentioned items 1 to 9, or the polycarbonate-based resin composition of the above-mentioned item 11 for a scratch-resistant application.

18. A use of the aromatic polycarbonate-based resin of any one of the above-mentioned items 1 to 9, or the polycarbonate-based resin composition of the above-mentioned item 12 for production of a resin window, a touch panel, an interior supply, an exterior supply, an interior part or an exterior part of a vehicle, a casing, an electric appliance, a building material, or OA equipment.

19. A use of a dihydric phenol-based compound represented by the following formula (ii) for production of an aromatic polycarbonate-based resin:

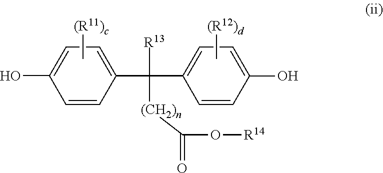

wherein in the formula (ii),
R¹¹ and R¹² each independently represent a halogen atom, or a group selected from the group consisting of: an alkyl group having 1 to 18 carbon atoms; an alkoxy group having 1 to 18 carbon atoms; a cycloalkyl group having 3 to 20 carbon atoms; a cycloalkoxy group having 6 to 20 carbon atoms; an alkenyl group having 2 to 10 carbon atoms; an aryl group having 6 to 14 carbon atoms; an aryloxy group having 6 to 14 carbon atoms; an aralkyl group having 7 to 20 carbon atoms; an aralkyloxy group having 7 to 20 carbon atoms; a nitro group; an aldehyde group; a cyano group; and a carboxyl group,
R¹³ represents a hydrogen atom, or a group selected from the group consisting of: an alkyl group having 1 to 5 carbon atoms; a cycloalkyl group having 3 to 20 carbon atoms; a cycloalkoxy group having 3 to 20 carbon atoms; an alkenyl group having 2 to 10 carbon atoms; and an aryl group having 6 to 14 carbon atoms,
R¹⁴ represents a saturated or unsaturated alicyclic group having 3 to 20 carbon atoms, or a 3- to 20-membered saturated or unsaturated heterocyclic group,
“c” and “d” each independently represent an integer of from 0 to 4, and
“n” represents an integer of from 0 to 20.

### Advantageous Effects of Invention

According to the present invention, the aromatic polycarbonate-based resin, the polycarbonate-based resin composition, and the molded article each of which achieves both of transparency and scratch resistance can be provided.

### BRIEF DESCRIPTION OF DRAWINGS

FIG. 1 is a ¹H-NMR chart of cyclohexyl diphenolate obtained in Synthesis Example 1.

FIG. 2 is a ¹H-NMR chart of cyclopentyl diphenolate obtained in Synthesis Example 2.

FIG. 3 is a ¹H-NMR chart of methyl diphenolate obtained in Synthesis Example 3.

FIG. 4 is a ¹H-NMR chart of an intermediate compound 2,2-bis(4-hydroxyphenyl)propanoic acid obtained in Synthesis Example 4.

FIG. 5 is a ¹H-NMR chart of cyclohexyl 2,2-bis(4-hydroxyphenyl)propanoate obtained in Synthesis Example 4.

FIG. 6 is a ¹H-NMR chart of a BPA-cyclohexyl diphenolate copolymer obtained in Production Example 1.

FIG. 7 is a ¹H-NMR chart of a BPA-cyclopentyl diphenolate copolymer obtained in Production Example 2.

FIG. 8 is a ¹H-NMR chart of a BPA-methyl diphenolate copolymer obtained in Production Example 3.

FIG. 9 is a ¹H-NMR chart of a BPA-cyclohexyl diphenolate copolymer obtained in Production Example 4.

FIG. 10 is a ¹H-NMR chart of a BPA-cyclohexyl diphenolate copolymer obtained in Production Example 5.

FIG. 11 is a ¹H-NMR chart of a BPA-cyclohexyl diphenolate copolymer obtained in Production Example 6.

FIG. 12 is a ¹H-NMR chart of a BPA-cyclohexyl diphenolate copolymer obtained in Production Example 7.

FIG. 13 is a ¹H-NMR chart of a BPA-cyclopentyl diphenolate copolymer obtained in Production Example 8.

FIG. 14 is a ¹H-NMR chart of a BPA-cyclopentyl diphenolate copolymer obtained in Production Example 9.

FIG. 15 is a ¹H-NMR chart of a BPA-cyclopentyl diphenolate copolymer obtained in Production Example 10.

FIG. 16 is a ¹H-NMR chart of a BPA-cyclohexyl 2,2-bis(4-hydroxyphenyl)propanoate copolymer obtained in Production Example 11.

FIG. 17 is a ¹H-NMR chart of a BPA-cyclohexyl 2,2-bis(4-hydroxyphenyl)propanoate copolymer obtained in Production Example 12.

FIG. 18 is a ¹H-NMR chart of a BPA-methyl diphenolate copolymer obtained in Production Example 13.

### DESCRIPTION OF EMBODIMENTS

An aromatic polycarbonate-based resin, a polycarbonate-based resin composition, and a molded article of the present invention are described in detail below. In this description, a specification considered to be preferred may be arbitrarily adopted, and it can be said that a combination of preferred specifications is more preferred. The term “XX to YY” as used herein means “XX or more and YY or less.”

1. Aromatic Polycarbonate-Based Resin

The aromatic polycarbonate-based resin of the present invention includes a repeating unit represented by the following formula (II):

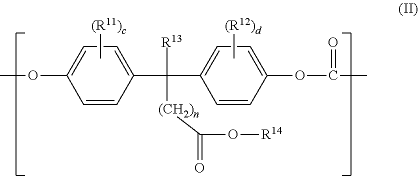

wherein in the formula (II),
R¹¹ and R¹² each independently represent a halogen atom, or a group selected from the group consisting of: an alkyl group having 1 to 18 carbon atoms; an alkoxy group having 1 to 18 carbon atoms; a cycloalkyl group having 4 to 20 carbon atoms; a cycloalkoxy group having 6 to 20 carbon atoms; an alkenyl group having 2 to 10 carbon atoms; an aryl group having 6 to 14 carbon atoms; an aryloxy group having 6 to 14 carbon atoms; an aralkyl group having 7 to 20 carbon atoms; an aralkyloxy group having 7 to 20 carbon atoms; a nitro group; an aldehyde group; a cyano group; and a carboxyl group,
R¹³ represents a hydrogen atom, or a group selected from the group consisting of: an alkyl group having 1 to 5 carbon atoms; a cycloalkyl group having 4 to 20 carbon atoms; a cycloalkoxy group having 6 to 20 carbon atoms; an alkenyl group having 2 to 10 carbon atoms; and an aryl group having 6 to 14 carbon atoms,
R¹⁴ represents a saturated or unsaturated alicyclic group having 3 to 20 carbon atoms, or a 3- to 20-membered saturated or unsaturated heterocyclic group,
“c” and “d” each independently represent an integer of from 0 to 4, and
“n” represents an integer of from 0 to 20.

Examples of the halogen atom that R¹¹ and R¹² in the formula (II) each independently represent include a fluorine atom, a chlorine atom, a bromine atom, and an iodine atom.

Examples of the alkyl group that R¹¹ and R¹² each independently represent include a methyl group, an ethyl group, a n-propyl group, an isopropyl group, various butyl groups (the term “various” means that a linear group and all kinds of branched groups are included, and the same holds true for the following), various pentyl groups, and various hexyl groups.

Examples of the alkoxy group that R¹¹ and R¹² each independently represent include alkoxy groups whose alkyl group moieties are the above-mentioned alkyl groups.

Examples of the cycloalkyl group that R¹¹ and R¹² each independently represent include a cyclopropyl group, a cyclobutyl group, a cyclopentyl group, a cyclohexyl group, and a cycloheptyl group.

Examples of the cycloalkyl group that R¹¹ and R¹² each independently represent include cycloalkoxy groups whose cycloalkyl group moieties are the above-mentioned cycloalkyl groups.

Examples of the alkenyl group that R¹¹ and R¹² each independently represent include an ethenyl group, a propenyl group, a butenyl group, a pentenyl group, and a hexenyl group.

Examples of the aryl group that R¹¹ and R¹² each independently represent include a phenyl group, a naphthyl group, a biphenyl group, and an anthryl group.

Examples of the aryloxy group that R¹¹ and R¹² each independently represent include aryloxy groups whose aryl group moieties are the above-mentioned aryl groups.

Examples of the aralkyl group that R¹¹ and R¹² each independently represent include a phenylmethyl group and a phenylethyl group.

Examples of the aralkyloxy group that R¹¹ and R¹² each independently represent include aralkyloxy groups whose aralkyl group moieties are the above-mentioned aralkyl groups.

Examples of the alkyl group represented by R¹³ in the formula (II) include a methyl group, an ethyl group, a n-propyl group, an isopropyl group, various butyl groups (the term “various” means that a linear group and all kinds of branched groups are included, and the same holds true for the following), various pentyl groups, and various hexyl groups.

Examples of the cycloalkyl group represented by R¹³ include a cyclopropyl group, a cyclobutyl group, a cyclopentyl group, a cyclohexyl group, and a cycloheptyl group.

Examples of the cycloalkoxy group represented by R¹³ include cycloalkoxy groups whose cycloalkyl group moieties are the above-mentioned cycloalkyl groups.

Examples of the alkenyl group represented by R¹³ include an ethenyl group, a propenyl group, a butenyl group, a pentenyl group, and a hexenyl group.

Examples of the aryl group represented by R¹³ include a phenyl group, a naphthyl group, a biphenyl group, and an anthryl group.

The saturated or unsaturated alicyclic group represented by R¹⁴ in the formula (II) has 3 to 20 carbon atoms, preferably 3 to 12 carbon atoms, more preferably 4 to 8 carbon atoms. Specific examples thereof include: cycloalkyl groups serving as saturated alicyclic groups, such as a cyclopropyl group, a cyclobutyl group, a cyclopentyl group, a cyclohexyl group, a cycloheptyl group, an adamantyl group, and a norbonyl group; and cycloalkenyl groups serving as unsaturated alicyclic groups, such as a cyclopropenyl group, a cyclobutenyl group, a cyclopentenyl group, a cyclohexenyl group, and a cycloheptenyl group. The unsaturated alicyclic group is free of an aromatic group.

The number of atoms for forming the ring of the heterocyclic group represented by R¹⁴ is from 3 to 20, and the ring has preferably 3 to 12 carbon atoms, more preferably 3 to 8 carbon atoms. The heterocyclic group is a cyclic group containing at least one heteroatom, for example, one, two, or three heteroatoms as ring-forming atoms. Specific examples of the heteroatom include a nitrogen atom, an oxygen atom, a sulfur atom, a silicon atom, a phosphorus atom, and a boron atom.

Examples of the heterocyclic group include a pyridinyl group, a pyrazinyl group, a pyrimidinyl group, a pyridazinyl group, a triazinyl group, an indolinyl group, a quinolinyl group, an acridinyl group, a pyrrolidinyl group, a dioxanyl group, a piperidinyl group, an oxiranyl group (epoxy group), an oxetanyl group, morpholidinyl group, a piperazinyl group, a carbazolyl group, a furanyl group, a thiophenyl group, an oxazolyl group, an oxadiazolyl group, a benzoxazolyl group, a thiazolyl group, a thiadiazolyl group, a benzothiazolyl group, a triazolyl group, an imidazolyl group, a benzimidazolyl group, and a furanyl group.

R¹⁴ represents preferably a saturated or unsaturated alicyclic group having 3 to 12 carbon atoms, or a 3- to 12-membered saturated or unsaturated heterocyclic group, more preferably a cycloalkyl group having 3 to 18 carbon atoms, still more preferably a cyclopentyl group or a cyclohexyl group.

“c” and “d” each independently represent an integer of from 0 to 4, preferably an integer of from 0 to 2, more preferably 0 or 1.

“n” represents an integer of from 0 to 20, preferably an integer of from 0 to 10, more preferably an integer of from 0 to 4, still more preferably 0, 1, 2, 3, or 4, still more preferably 2.

As another aspect, “n” represents preferably an integer of from 1 to 10, more preferably an integer of from 1 to 4.

In a preferred aspect of the formula (II), from the viewpoint of achieving both of transparency and scratch resistance, R¹⁴ represents a cyclopentyl group or a cyclohexyl group, and “n” represents 2. In addition, “c” and “d” each preferably represent 0, and R¹³ represents preferably an alkyl group having 1 to 3 carbon atoms, more preferably a methyl group.

In the aromatic polycarbonate-based resin, the repeating units each represented by the formula (II) may be used alone or in combination thereof.

The aromatic polycarbonate-based resin may further include a repeating unit represented by the following formula (I):

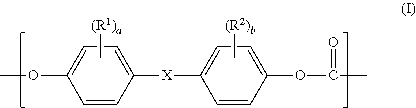

wherein in the formula (I),
R¹ and R² each independently represent a halogen atom, or a group selected from the group consisting of: an alkyl group having 1 to 18 carbon atoms; an alkoxy group having 1 to 18 carbon atoms; a cycloalkyl group having 6 to 20 carbon atoms; a cycloalkoxy group having 6 to 20 carbon atoms; an alkenyl group having 2 to 10 carbon atoms; an aryl group having 6 to 14 carbon atoms; an aryloxy group having 6 to 14 carbon atoms; an aralkyl group having 7 to 20 carbon atoms; an aralkyloxy group having 7 to 20 carbon atoms; a nitro group; an aldehyde group; a cyano group; and a carboxyl group,
X represents a single bond, an alkylene group having 1 to 8 carbon atoms, an alkylidene group having 2 to 8 carbon atoms, a cycloalkylene group having 5 to 15 carbon atoms, a cycloalkylidene group having 5 to 15 carbon atoms, an aralkyl group having 7 to 20 carbon atoms, —S—, —SO—, —SO₂—, —O—, or —CO—, and
“a” and “b” each independently represent an integer of from 0 to 4.

Examples of the halogen atom that R¹ and R² in the formula (I) each independently represent include a fluorine atom, a chlorine atom, a bromine atom, and an iodine atom.

Examples of the alkyl group that R¹ and R² each independently represent include a methyl group, an ethyl group, a n-propyl group, an isopropyl group, various butyl groups (the term “various” means that a linear group and all kinds of branched groups are included, and the same holds true for the following), various pentyl groups, and various hexyl groups.

Examples of the alkoxy group that R¹ and R² each independently represent include alkoxy groups whose alkyl group moieties are the above-mentioned alkyl groups.

Examples of the cycloalkyl group that R¹ and R² each independently represent include a cyclopropyl group, a cyclobutyl group, a cyclopentyl group, a cyclohexyl group, and a cycloheptyl group.

Examples of the cycloalkyl group that R¹ and R² each independently represent include cycloalkoxy groups whose cycloalkyl group moieties are the above-mentioned cycloalkyl groups.

Examples of the alkenyl group that R¹ and R² each independently represent include an ethenyl group, a propenyl group, a butenyl group, a pentenyl group, and a hexenyl group.

Examples of the aryl group that R¹ and R² each independently represent include a phenyl group, a naphthyl group, a biphenyl group, and an anthryl group.

Examples of the aryloxy group that R¹ and R² each independently represent include aryloxy groups whose aryl group moieties are the above-mentioned aryl groups.

Examples of the aralkyl group that R¹ and R² each independently represent include a phenylmethyl group and a phenylethyl group.

Examples of the aralkyloxy group that R¹ and R² each independently represent include aralkyloxy groups whose aralkyl group moieties are the above-mentioned aralkyl groups.

The alkylene group represented by X has 1 to 8 carbon atoms, preferably 1 to 5 carbon atoms. Specific examples thereof include a methylene group, an ethylene group, a trimethylene group, a tetramethylene group, and a hexamethylene group.

Examples of the alkylidene group represented by X include an ethylidene group and an isopropylidene group.

The cycloalkylene group represented by X has 5 to 15 carbon atoms, preferably 5 to 10 carbon atoms. Specific examples thereof include a cyclopentanediyl group, a cyclohexanediyl group, and a cyclooctanediyl group.

Examples of the arylene group represented by X include a phenylene group, a naphthylene group, a biphenylene group, and a tetraphenyl group.

The cycloalkylidene group represented by X has 5 to 15 carbon atoms, preferably 5 to 10 carbon atoms. Specific examples thereof include a cyclohexylidene group, a 3,5,5-trimethylcyclohexylidene group, and a 2-adamantylidene group.

Examples of the aryl moiety of the aralkyl group (arylalkylene group) represented by X include aryl groups each having 6 to 14 ring-forming carbon atoms, such as a phenyl group, a naphthyl group, a biphenyl group, and an anthryl group.

A case in which X represents an isopropylidene group, a cyclohexylidene group, or a 3,5,5-trimethylcyclohexylidene group among those described above is preferred because a molded body of the aromatic polycarbonate-based resin can achieve both of surface hardness and mechanical properties.

“a” and “b” each independently represent an integer of from 0 to 4, preferably from 0 to 2, more preferably 0 or 1.

Among such resins, a resin in which “a” and “b” each represent 0, and X represents a single bond or an alkylene group having 1 to 8 carbon atoms, or a resin in which “a” and “b” each represent 0, and X represents an alkylidene group, in particular, an isopropylidene group is suitable.

In addition, as another aspect, a resin in which “a” and “b” each represent 1, and X represents a single bond or an alkylene group having 1 to 8 carbon atoms, or a resin in which “a” and “b” each represent 1, and X represents an alkylidene group, in particular, an isopropylidene group is preferred because the molded body of the aromatic polycarbonate-based resin can achieve both of the surface hardness and the mechanical properties.

Specific examples of the repeating unit represented by the formula (I) include repeating units represented by the following formulae (I-i) to (I-iv).

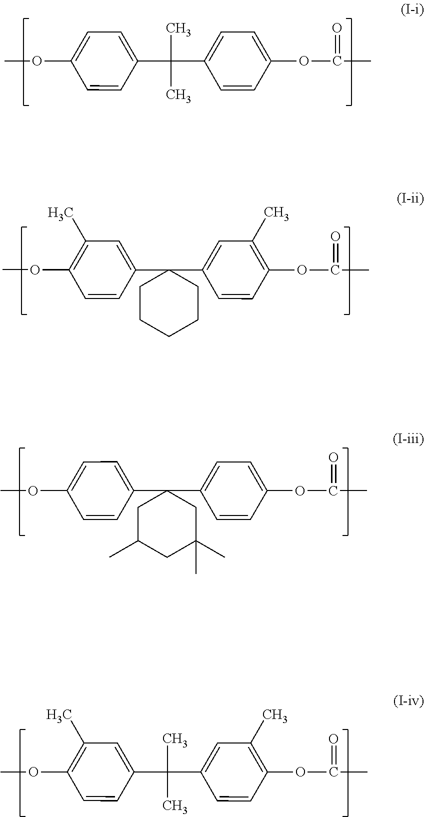

In the aromatic polycarbonate-based resin, the repeating units each represented by the formula (I) may be used alone or in combination thereof. Specifically, for example, the following aspects are given: an aspect in which the resin is formed only of the repeating unit represented by the formula (I-i); and an aspect in which the resin is formed of a combination of the repeating unit represented by the formula (I-i), and one or more kinds selected from the group consisting of the repeating units represented by the formulae (I-ii) to (I-iv). Such aromatic polycarbonate-based resin can be easily produced by an interfacial polymerization method in which a polycarbonate oligomer to be described later is produced in advance.

When the aromatic polycarbonate-based resin includes the repeating unit represented by the formula (I), the aromatic polycarbonate-based resin is an aromatic polycarbonate-based copolymer including the repeating unit represented by the formula (I) and the repeating unit represented by the formula (II).

In the aromatic polycarbonate-based resin, a molar ratio ((I):(II)) between the repeating unit represented by the formula (I) and the repeating unit represented by the formula (II) is preferably from 0:100 to 99.5:0.5, more preferably from 0.5:99.5 to 99.5:0.5, still more preferably from 0.5:99.5 to 99:1, still further more preferably from 0.5:99.5 to 94:6, most preferably from 0.5:99.5 to 92:8.

The molar ratio ((I):(II)) between the repeating unit represented by the formula (I) and the repeating unit represented by the formula (II) is preferably from 60:40 to 99.5:0.5, more preferably from 70:30 to 99:1, still more preferably from 80:20 to 98:2 among those molar ratios.

The molar ratio between the repeating unit represented by the formula (I) and the repeating unit represented by the formula (II) in the aromatic polycarbonate-based resin is calculated by nuclear magnetic resonance (NMR) measurement. Specifically, ¹H-NMR measurement is performed, and the ratio is calculated from the integrated values of a peak derived from the repeating unit represented by the formula (I) and a peak derived from the repeating unit represented by the formula (II).

The aromatic polycarbonate-based resin of the present invention may include a constituent unit except the repeating unit represented by the formula (I) and the repeating unit represented by the formula (II). Examples of such unit include a terminal structure derived from an end-capping agent to be described later and a silicon atom-containing constituent unit.

The viscosity-average molecular weight of the aromatic polycarbonate-based resin is preferably from 10,000 to 100,000, more preferably from 10,000 to 80,000, still more preferably from 15,000 to 30,000, still more preferably from 17,000 to 25,000 in terms of mechanical characteristics and moldability.

In the present invention, the viscosity-average molecular weight (Mv) is calculated from the following Schnell's equation after the determination of a limiting viscosity [η] through the measurement of the viscosity of a methylene chloride solution (concentration: g/L) at 20° C. with an Ubbelohde-type viscometer.

$$[ η ] = 1.23 \times {10}^{- 5} {Mv}^{0.83}$$

The molded body of the aromatic polycarbonate-based resin of the present invention can achieve both of excellent transparency and excellent scratch resistance.

The scratch resistance may be evaluated by scratch hardness (pencil method). The scratch hardness (pencil method) of the molded body of the aromatic polycarbonate-based resin evaluated in conformity with JIS K5600-5-4:1999 is preferably F or more.

The transparency may be evaluated by a total light transmittance. The total light transmittance of the molded body of the aromatic polycarbonate-based resin when the molded body has a thickness of 1.5 mm, the total light transmittance being measured in conformity with ASTM D1003, is preferably 87% or more, more preferably 88% or more, still more preferably 89% or more.

### 2. Method of Producing Aromatic Polycarbonate-Based Resin (Dihydric Phenol-Based Compound)

The above-mentioned aromatic polycarbonate-based resin may be suitably produced by using a dihydric phenol-based compound (a) represented by the following formula (ii). The repeating unit represented by the formula (II) of the polycarbonate-based resin is derived from the dihydric phenol-based compound (a).

Accordingly, the present invention also provides a use of the dihydric phenol-based compound (a) represented by the following formula (ii) for production of an aromatic polycarbonate-based resin:

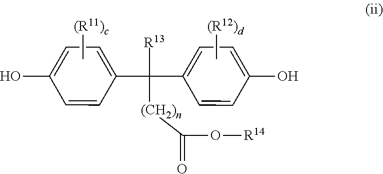

wherein
in the formula (ii), R¹¹, R¹², R¹³, R¹⁴, “c”, “d”, and “n” are as defined above, and preferred examples thereof are also the same as those described above.

Preferred specific examples of the dihydric phenol-based compound (a) include cyclohexyl diphenolate represented by the following formula (ii-1) and cyclopentyl diphenolate represented by the following formula (ii-2).

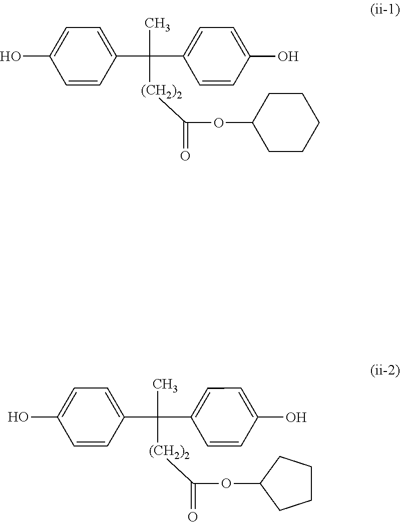

The dihydric phenol-based compound (a) may be produced by, for example, causing a carboxylic acid compound (a-x) represented by the following formula (ii-x) and an alcohol compound (a-y) represented by the following formula (ii-y) to react with each other in the presence of an acid catalyst as required:

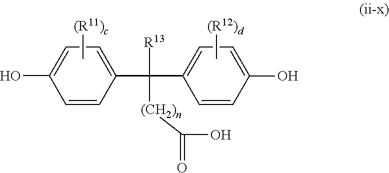

wherein
in the formula (ii-x), R¹¹, R¹², R¹³, “c”, “d”, and “n” are as defined above, and preferred examples thereof are also the same as those described above;

R¹⁴—OH (ii-y)
wherein
in the formula (ii-y), R¹⁴ is as defined above, and preferred examples thereof are also the same as those described above.

### (Method of Producing Aromatic Polycarbonate-Based Resin)

The aromatic polycarbonate-based resin may be produced by a known method of producing a polycarbonate-based resin as long as the dihydric phenol-based compound (a) represented by the formula (ii) is used as a dihydric phenol-based compound. Examples of the method of producing a polycarbonate-based resin include:
(i) an interfacial polymerization method (phosgene method) including causing the dihydric phenol-based compound and phosgene to react with each other in the presence of an organic solvent inert to the reaction and an aqueous alkali solution, and then adding a polymerization catalyst, such as a tertiary amine or a quaternary ammonium salt, to polymerize the resultant;
(ii) a melt polymerization method (ester exchange method) including subjecting the dihydric phenol-based compound and a carbonic acid diester to an ester exchange reaction under a molten state in which no solvent is used through the addition of a basic catalyst; and
(iii) a pyridine method including dissolving the dihydric phenol-based compound in pyridine or a mixed solution containing pyridine and an inert solvent, and introducing phosgene into the solution to directly produce the resin.

A molecular weight modifier (an end-capping agent), a branching agent, or the like is used as required in the reaction.

Among them, the following production method is preferred:
a method of producing an aromatic polycarbonate-based resin, including a step of subjecting the dihydric phenol-based compound and a polycarbonate oligomer to interfacial polycondensation in the presence of a water-insoluble organic solvent and an aqueous solution of an alkali compound, wherein the dihydric phenol-based compound contains the dihydric phenol-based compound (a) represented by the formula (ii).

Specifically, in the case of the interfacial polymerization method, the aromatic polycarbonate-based resin may be produced by: dissolving a polycarbonate oligomer produced in advance to be described later in a water-insoluble organic solvent (e.g., methylene chloride); adding a solution of a dihydric phenol-based compound in an aqueous alkali compound (e.g., aqueous sodium hydroxide) to the solution; and subjecting the mixture to an interfacial polycondensation reaction through use of a tertiary amine (e.g., triethylamine) or a quaternary ammonium salt (e.g., trimethylbenzylammonium chloride) as a polymerization catalyst in the presence of an end-capping agent (a monohydric phenol such as p-tert-butylphenol) as required. In addition, in the case of the interfacial polymerization method, the aromatic polycarbonate-based resin may be produced by copolymerizing a dihydric phenol and phosgene, a carbonic acid ester, or a chloroformate.

The polycarbonate oligomer may be produced by a reaction between a dihydric phenol-based compound and a carbonate precursor, such as phosgene or triphosgene, in an organic solvent, such as methylene chloride, chlorobenzene, or chloroform. When the polycarbonate oligomer is produced by using an ester exchange method, the oligomer may be produced by a reaction between the dihydric phenol-based compound and a carbonate precursor such as diphenyl carbonate.

The dihydric phenol contains the dihydric phenol-based compound (a) represented by the following formula (ii) from which the repeating unit represented by the formula (II) is derived. The dihydric phenol preferably further contains a dihydric phenol-based compound (b) represented by the following formula (i) from which the repeating unit represented by the formula (I) is derived.

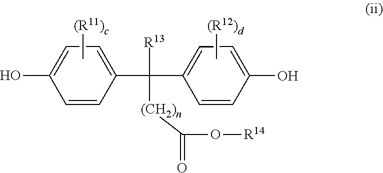

wherein
in the formula (ii), R¹¹, R¹², R¹³, R¹⁴, “c”, “d”, and “n” are as defined above, and preferred examples thereof are also the same as those described above.

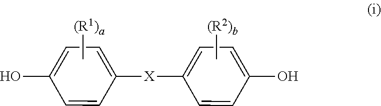

wherein
in the formula (i), R¹, R², X, “a”, and “b” are as defined above, and preferred examples thereof are also the same as those described above.

Examples of the dihydric phenol-based compound (b) include: bis(hydroxyphenyl)alkane-based dihydric phenols, such as 2,2-bis(4-hydroxyphenyl)propane [bisphenol A (BPA)], bis(4-hydroxyphenyl)methane, 1,1-bis(4-hydroxyphenyl)ethane, and 2,2-bis(4-hydroxy-3,5-dimethylphenyl)propane; 4,4′-dihydroxydiphenyl; bis(4-hydroxyphenyl)cycloalkanes; bis(4-hydroxyphenyl) oxide; bis(4-hydroxyphenyl) sulfide; bis(4-hydroxyphenyl) sulfone; bis(4-hydroxyphenyl) sulfoxide; and bis(4-hydroxyphenyl) ketone. Those dihydric phenol-based compounds may be used alone or as a mixture thereof.

Among them, bis(hydroxyphenyl)alkane-based dihydric phenols are preferred, and bisphenol A is more preferred.

In a preferred production method, only the dihydric phenol-based compound (b) may be used as a dihydric phenol-based compound for producing the polycarbonate oligomer. In this case, in the step of the interfacial polycondensation reaction, the dihydric phenol-based compound (a) and the dihydric phenol-based compound (b) are used in combination, or only the dihydric phenol-based compound (a) is used.

In order to adjust the molecular weight of the aromatic polycarbonate-based resin to be obtained, an end-capping agent (molecular weight modifier) may be used. Examples of the end-capping agent may include monohydric phenols, such as phenol, p-cresol, p-tert-butylphenol, p-tert-octylphenol, p-cumylphenol, p-nonylphenol, m-pentadecylphenol, and p-tert-amylphenol. Those monohydric phenols may be used alone or in combination thereof.

The aromatic polycarbonate-based resin of the present invention may be suitably used in an scratch-resistant application because a molded body thereof can achieve both of excellent transparency and excellent scratch resistance.

### 3. Polycarbonate-Based Resin Composition

The polycarbonate-based resin composition of the present invention includes the above-mentioned aromatic polycarbonate-based resin, and as required, any other component.

Examples of the other component may include additives, such as a hydrolysis-resistant agent, an antioxidant, a UV absorber, a flame retardant, a flame-retardant aid, a reinforcing material, a filler, an elastomer for an impact resistance improvement, a pigment, and a dye.

The polycarbonate-based resin composition may include an antioxidant from, for example, the viewpoint that its oxidative deterioration at the time of melting can be prevented, and hence its coloring or the like due to the oxidative deterioration is prevented.

The content of the antioxidant is preferably 0.001 part by mass or more and 0.5 part by mass or less, more preferably 0.01 part by mass or more and 0.3 part by mass or less, still more preferably 0.02 part by mass or more and 0.2 part by mass or less with respect to 100 parts by mass of the content of the aromatic polycarbonate-based resin. When the content of the antioxidant falls within the above-mentioned ranges, a sufficient antioxidant action is obtained, and mold contamination at the time of the molding of the resin composition can be suppressed.

A method of producing the polycarbonate-based resin composition of the present invention is not particularly limited as long as the method includes a step of mixing the aromatic polycarbonate-based resin and any other optional component. The composition may be produced by, for example, mixing the aromatic polycarbonate-based resin and any other optional component with a mixer or the like, and melting and kneading the mixture. The melting and kneading may be performed by a typically used method such as a method including using, for example, a ribbon blender, a Henschel mixer, a Banbury mixer, a drum tumbler, a single-screw extruder, a twin-screw extruder, a co-kneader, or a multi-screw extruder. A heating temperature at the time of the melting and kneading is appropriately selected from the range of, for example, from 150° C. to 300° C., preferably from about 220° C. to about 300° C.

The polycarbonate-based resin composition of the present invention may be suitably used in a scratch-resistant application because a molded body thereof can achieve both of excellent transparency and excellent scratch resistance.

The scratch-resistant application is, for example, a structural body whose outer surface is formed of the polycarbonate-based resin composition, and more specific examples thereof include a resin window, a touch panel, an interior supply, an exterior supply, an interior part or an exterior part of a vehicle, a casing, an electric appliance, a building material, and OA equipment. The polycarbonate-based resin composition of the present invention may be suitably used for producing the above-mentioned articles.

### 4. Molded Body

The molded body of the present invention includes the above-mentioned polycarbonate-based resin composition. The molded body may be produced through use of a melt-kneaded product of the polycarbonate-based resin composition or a pellet thereof obtained through melting and kneading as a raw material by an injection molding method, an injection compression molding method, an extrusion molding method, a blow molding method, a press molding method, a vacuum molding method, an expansion molding method, or the like. In particular, the molded body is preferably produced through use of a pellet obtained through melting and kneading by an injection molding method or an injection compression molding method.

The thickness of the molded body may be arbitrarily set in accordance with applications, and particularly when the transparency of the molded body is required, the thickness is preferably from 0.2 mm to 4.0 mm, more preferably from 0.3 mm to 3.0 mm, still more preferably from 0.3 mm to 2.0 mm. When the thickness of the molded body is 0.2 mm or more, no warping occurs, and hence satisfactory mechanical strength is obtained. In addition, when the thickness of the molded body is 4.0 mm or less, high transparency is obtained.

The molded body formed of the polycarbonate-based resin composition of the present invention may be suitably used as, for example, a resin window, a touch panel, an interior supply, an exterior supply, an interior part or an exterior part of a vehicle, a casing, an electric appliance, or a building material.

### EXAMPLES

Examples of the present invention are further described. The present invention is by no means limited by these examples. Measurement and evaluations in the respective examples were performed by the following methods.

### 1. Measurement of Viscosity-Average Molecular Weight (Mv)

A viscosity-average molecular weight (Mv) was calculated from the following equation (Schnell's equation) after the determination of a limiting viscosity [η] through the measurement of the viscosity of a methylene chloride solution (concentration: g/L) at 20° C. with an Ubbelohde-type viscometer.

$$[ η ] = 1.23 \times {10}^{- 5} {Mv}^{0.83}$$

### 2. ¹H-NMR Measurement Conditions

Nuclear magnetic resonance (NMR) apparatus: “Asend 500” manufactured by Bruker Japan K. K.
Probe: 5 mmp TCI cryoprobe
Observed range: 20 ppm
Observation center: 6.175 ppm
Pulse repetition time: 10 seconds
Flip angle: 300
NMR sample tube: 5 mmφ
Sample amount: 50 mg
Solvent: deuterochloroform containing tetramethylsilane (TMS)
Measurement temperature: 25° C.
Number of scans: 256 times
Chemical shift correction: the peak of TMS is set to a reference of 0 ppm.

### 3. Determination of Composition Ratio of Aromatic Polycarbonate-based Resin

The ¹H-NMR of a sample dissolved in deuterochloroform containing TMS was measured with “Asend 500” manufactured by Bruker Japan K. K. under the same measurement conditions as those described above, and the structure of an aromatic polycarbonate-based resin was assigned.

Specifically, the integrated values (i) to (iii) of the following peaks were determined.
In case of BPA-Cyclohexyl Diphenolate Copolymer
(i) An integrated value obtained by summing those of a phenyl group of a bisphenol A (BPA) moiety, a phenyl group of a cyclohexyl diphenolate moiety, and a phenyl group of a p-tert-butylphenol (PTBP) moiety observed at a δ of from around 6.8 to around 7.5
(ii) The integrated value of a methine group of the cyclohexyl diphenolate moiety observed at a δ of from around 4.6 to around 4.8
(iii) The integrated value of a methyl group of the PTBP moiety observed at a δ of from around 1.30 to around 1.33
In case of BPA-Cyclopentyl Diphenolate Copolymer
(i) An integrated value obtained by summing those of a phenyl group of a BPA moiety, a phenyl group of a cyclopentyl diphenolate moiety, and a phenyl group of a PTBP moiety observed at a δ of from around 6.8 to around 7.5
(ii) The integrated value of a methine group of the cyclopentyl diphenolate moiety observed at a δ of from around 5.05 to around 5.15
(iii) The integrated value of a methyl group of the PTBP moiety observed at a δ of from around 1.30 to around 1.33
In case of BPA-Cyclohexyl 2,2-Bis(4-hydroxyphenyl)propanoate Copolymer
(i) An integrated value obtained by summing those of a phenyl group of a bisphenol A (BPA) moiety, a phenyl group of a cyclohexyl 2,2-bis(4-hydroxyphenyl)propanoate moiety, and a phenyl group of a p-tert-butylphenol (PTBP) moiety observed at a δ of from around 6.8 to around 7.5
(ii) The integrated value of a methine group of the cyclohexyl 2,2-bis(4-hydroxyphenyl)propanoate moiety observed at a δ of from around 4.75 to around 4.95
(iii) The integrated value of a methyl group of the PTBP moiety observed at a δ of from around 1.30 to around 1.33
In case of BPA-Methyl Diphenolate Copolymer
(i) An integrated value obtained by summing those of a phenyl group of a BPA moiety, a phenyl group of a methyl diphenolate moiety, and a phenyl group of a PTBP moiety observed at a δ of from around 6.8 to around 7.5
(ii) The integrated value of a methyl group of the methyl diphenolate moiety observed at a δ of from around 3.4 to around 3.8
(iii) The integrated value of a methyl group of the PTBP moiety observed at a δ of from around 1.25 to around 1.35

The contents of the respective repeating units in the aromatic polycarbonate-based resin were determined from the following equations on the basis of the above-mentioned integrated values in consideration of the number of protons.
In cases of Cyclohexyl Diphenolate, Cyclopentyl Diphenolate, and Cyclohexyl 2,2-Bis(4-hydroxyphenyl)propanoate

$$a = ( ( i ) - ( ii ) \times 8 - ( iii ) \times 4 / 9 ) / 8$$

b=(ii)

$$c = ( iii ) / 9 \\ T = a + b + c$$

BPA copolymerization composition ratio (mol %) A=(a/T×100)×(100/(100−c))
Diphenolic acid ester copolymerization composition ratio (mol %) B=

$$( b / T \times 100 ) \times ( 100 / ( 100 - c ) )$$

In case of Methyl Diphenolate

$$a = ( ( i ) - ( ii ) \times 8 - ( iii ) \times 4 / 9 ) / 8 \\ b = ( ii ) / 3 \\ c = ( iii ) / 9 \\ T = a + b + c$$

BPA copolymerization composition ratio (mol %) A=(a/T×100)×(100/(100−c))
Diphenolic acid ester copolymerization composition ratio (mol %) B=(b/T×100)×(100/(100−c))

### Synthesis Example 1 (Synthesis of Cyclohexyl Diphenolate)

620 Milliliters of cyclohexanol, 111 g (388 mmol) of diphenolic acid, and 5.69 g (58.0 mmol) of sulfuric acid were loaded into a 1-liter flask to provide a reaction liquid, and a stirrer chip, a temperature gauge, and a reflux tube were set in the flask. The temperature of the reaction liquid was increased to 80° C. with an oil bath, and the liquid was stirred with a magnetic stirrer for 19 hours. The disappearance of diphenolic acid was recognized by thin-layer chromatography (TLC), and then the temperature of the reaction liquid was returned to room temperature. 600 Milliliters of toluene was loaded into the reaction liquid, and the mixture was washed with 800 mL of baking soda water (saturated aqueous solution of sodium hydrogen carbonate) twice and with 800 mL of brine (saturated salt solution) once. The organic phase was dried with anhydrous sodium sulfate, and was then concentrated under reduced pressure to provide 316 g of a pale brown liquid as a crude product. The resultant crude product was purified with a silica gel column (neutral silica gel: 1.05 kg, solvent: heptane/ethyl acetate=4/1) to provide 263 g of a pale yellow liquid. The resultant pale yellow liquid was subjected to azeotropy with a mixed solvent containing acetonitrile and water at 2/1 (300 g) eight times. The precipitated solid was recovered by filtration under reduced pressure, and was subjected to suspension washing with 500 mL of hexane twice. The resultant solid was dried under reduced pressure at 40° C. for 12 hours to provide 113 g of a white solid of cyclohexyl diphenolate.

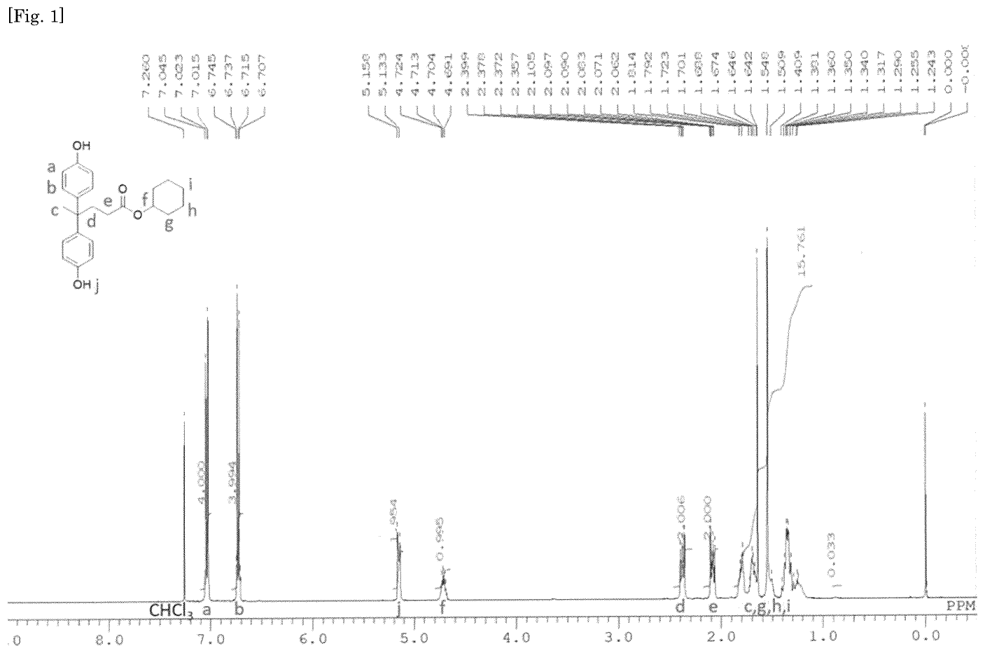

The ¹H-NMR chart of the resultant compound is shown in FIG. 1.

### Synthesis Example 2 (Synthesis of Cyclopentyl Diphenolate)

694 Milliliters of cyclopentanol, 124 g (434 mmol) of diphenolic acid, and 6.37 g (65.0 mmol) of sulfuric acid were loaded into a 1-liter flask to provide a reaction liquid, and a stirrer chip, a temperature gauge, and a reflux tube were set in the flask. The temperature of the reaction liquid was increased to 80° C. with an oil bath, and the liquid was stirred with a magnetic stirrer for 22 hours. The disappearance of diphenolic acid was recognized by thin-layer chromatography (TLC), and then the temperature of the reaction liquid was returned to room temperature. 600 mL of ethyl acetate was loaded into the reaction liquid, and the mixture was washed with 600 mL of baking soda water (saturated aqueous solution of sodium hydrogen carbonate) twice and with 600 mL of brine (saturated salt solution) once. The organic phase was dried with anhydrous sodium sulfate, and was then concentrated under reduced pressure to provide 172 g of a pale brown liquid as a crude product. The resultant crude product was dissolved in 350 mL of a mixed solvent containing hexane and ethyl acetate at 9/1, and the solution was left at rest at room temperature all night. 108.2 Grams of cyclopentyl diphenolate serving as a target product was obtained by recrystallization.

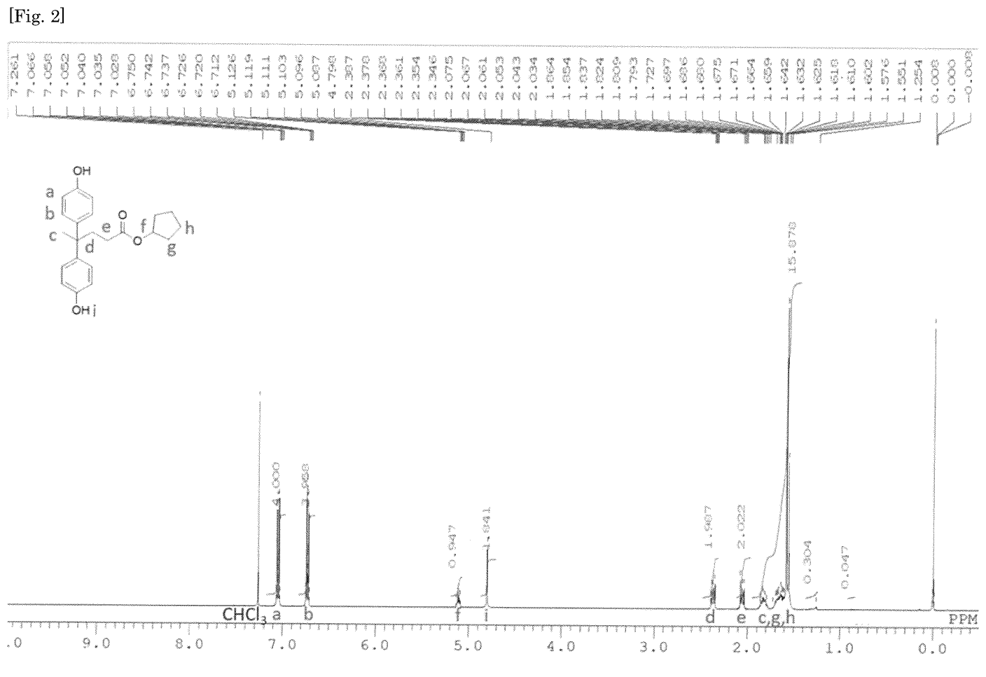

The ¹H-NMR chart of the resultant compound is shown in FIG. 2.

### Synthesis Example 3 (Synthesis of Methyl Diphenolate)

500 mL of methanol was loaded into a 1-liter flask, and then 50.0 g of diphenolic acid was dissolved therein. Next, 2.5 mL of sulfuric acid was added to the solution, and the mixture was refluxed for 5 hours. After that, the reaction solution was left standing to cool to room temperature, and was concentrated with a rotary evaporator. 200 mL of ethyl acetate was added to the concentrate, and the mixture was washed with 100 mL of baking soda water (saturated aqueous solution of sodium hydrogen carbonate) three times and with 100 mL of pure water twice. The recovered organic phase was concentrated with the rotary evaporator, and was then dried under reduced pressure to provide 57.14 g of a light yellow solid of methyl diphenolate.

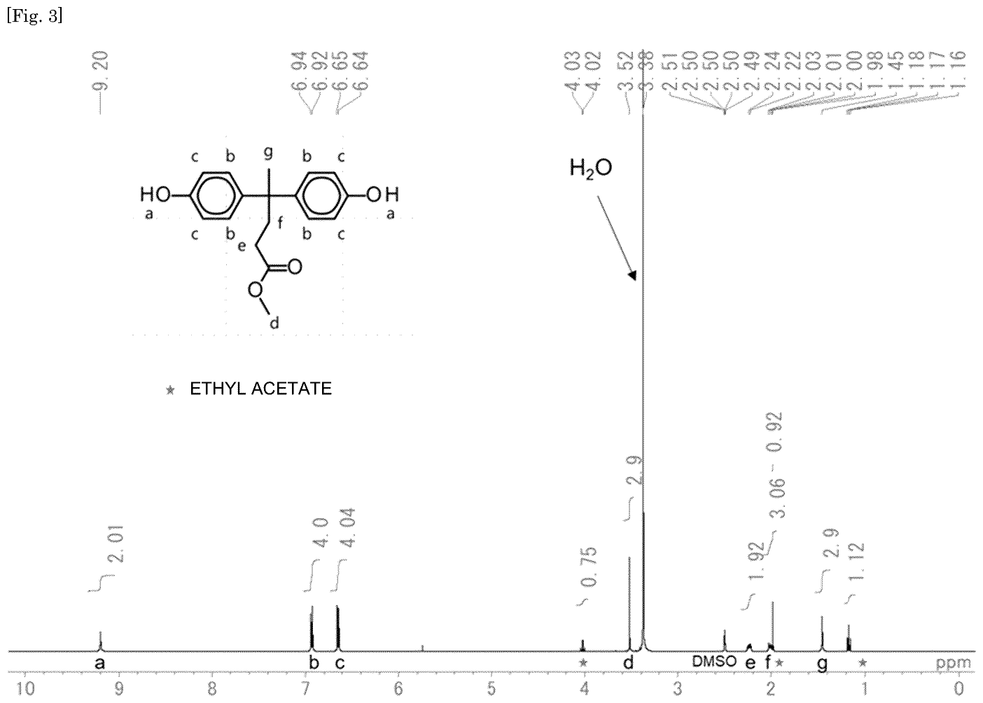

The ¹H-NMR chart of the resultant compound is shown in FIG. 3.

### Synthesis Example 4 (Synthesis of Cyclohexyl 2,2-Bis(4-hydroxyphenyl)propanoate)

131 Grams of phenol, 60.3 g of pyruvic acid, and 45.6 mL of ion-exchanged water were loaded into a 1-liter four-necked flask including a stirrer bar and a temperature gauge, and the flask was cooled with ice. 112 Grams of 95% sulfuric acid was dropped into the mixture over 50 minutes, and then the temperature of the whole was increased to room temperature, followed by stirring for 14 hours. 1 Liter of diethyl ether was loaded into the reaction liquid, and the mixture was washed with 1 L of ion-exchanged water once. The organic phase was extracted with 1 L of 0.1 mol/L aqueous sodium hydroxide twice. The pH of the extracted organic phase was adjusted to 2 with a 1 mol/L aqueous solution of hydrochloric acid, and the organic phase was extracted with 1 L of diethyl ether twice. The organic phase was dried with sodium sulfate, and was then dried under reduced pressure to provide 139 g of a pale brown solid of 2,2-bis(4-hydroxyphenyl)propanoic acid as an intermediate compound.

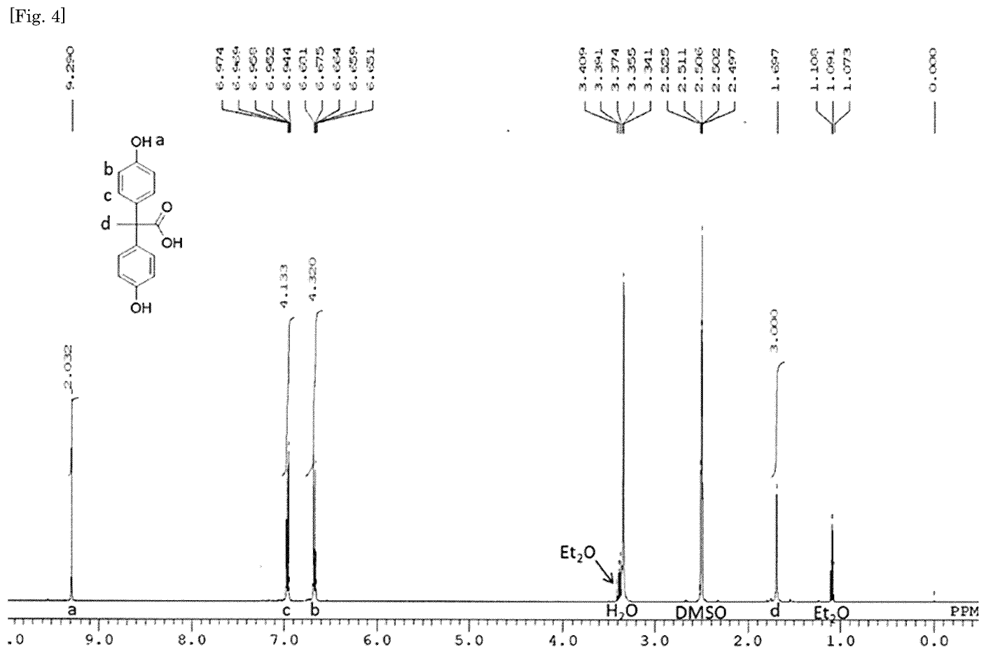

The ¹H-NMR chart of the resultant intermediate compound is shown in FIG. 4.

Subsequently, 2.01 L of cyclohexanol, 130 g of 2,2-bis(4-hydroxyphenyl)propanoic acid, and 27.7 g of 95% sulfuric acid were loaded into a 5-liter four-necked flask including a stirring blade, a temperature gauge, and a reflux tube, and the temperature of the reaction liquid was increased to 100° C., followed by stirring for 15 days. The reaction liquid was cooled to room temperature, and was then diluted twofold with diethyl ether, followed by washing with baking soda water twice, with ion-exchanged water once, and with a saturated salt solution once. The organic phase was isolated, was dried with magnesium sulfate, and was concentrated under reduced pressure to provide 815 g of a crude product. The crude product was purified by silica gel chromatography (neutral silica gel: 5.0 kg, solvent: the concentration gradient “chloroform/ethyl acetate” was changed from 1/0 vol % to 0/1 vol %) three times. Next, the purified product was purified by recrystallization (chloroform/ethyl acetate=1/1 vol %) to provide 52.9 g of a white solid of cyclohexyl 2,2-bis(4-hydroxyphenyl)propanoate.

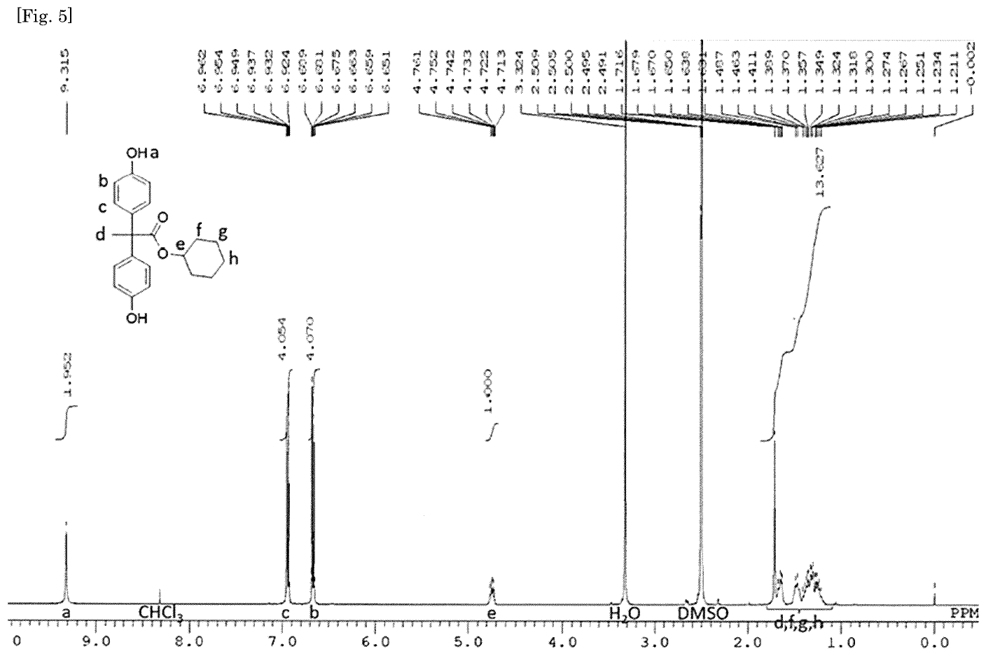

The ¹H-NMR chart of the resultant compound is shown in FIG. 5.

### Synthesis Example 5 (Polycarbonate Oligomer Synthesis (1))

Sodium dithionite was added to 5.6 mass % aqueous sodium hydroxide so that its concentration became 2,000 ppm with respect to bisphenol A (BPA) to be dissolved later. BPA was dissolved therein so that the concentration of BPA was 13.5 mass %. Thus, a solution of BPA in aqueous sodium hydroxide was prepared.

The solution of BPA in aqueous sodium hydroxide, methylene chloride, and phosgene were continuously passed through a tubular reactor having an inner diameter of 6 mm and a tube length of 30 m at flow rates of 40 L/hr, 15 L/hr, and 4.0 kg/hr, respectively. The tubular reactor had a jacket portion and the temperature of the reaction liquid was kept at 40° C. or less by passing cooling water through the jacket. The reaction liquid that had exited the tubular reactor was continuously introduced into a baffled vessel-type reactor provided with a sweptback blade and having an internal volume of 40 L. The solution of BPA in aqueous sodium hydroxide, 25 mass % aqueous sodium hydroxide, water, and a 1 mass % aqueous solution of triethylamine were further added to the reactor at flow rates of 2.8 L/hr, 0.07 L/hr, 17 L/hr, and 0.64 L/hr, respectively, to perform a reaction. An aqueous phase was separated and removed by continuously taking out the reaction liquid overflowing the vessel-type reactor and leaving the reaction liquid at rest. Then, a methylene chloride phase was collected.

The solution of a polycarbonate oligomer (PCO) in methylene chloride (PCO solution (a)) thus obtained had a concentration of 341 g/L and a chloroformate group concentration of 0.71 mol/L.

### Synthesis Example 6 (Polycarbonate Oligomer Synthesis (2))

Sodium dithionite was added to 5.6 mass % aqueous sodium hydroxide so that its concentration became 2,000 ppm with respect to bisphenol A (BPA) to be dissolved later. BPA was dissolved therein so that the concentration of BPA was 13.5 mass %. Thus, a solution of BPA in aqueous sodium hydroxide was prepared.

The solution of BPA in aqueous sodium hydroxide, methylene chloride, and phosgene were continuously passed through a tubular reactor having an inner diameter of 6 mm and a tube length of 30 m at flow rates of 40 L/hr, 18 L/hr, and 4.5 kg/hr, respectively. The tubular reactor had a jacket portion and the temperature of the reaction liquid was kept at 40° C. or less by passing cooling water through the jacket. An aqueous phase was separated and removed by continuously taking out the reaction liquid that had exited the tubular reactor and leaving the reaction liquid at rest. Then, a methylene chloride phase was collected.

The solution of a polycarbonate oligomer (PCO) in methylene chloride (PCO solution (b)) thus obtained had a concentration of 308 g/L and a chloroformate group concentration of 0.94 mol/L.

### Production Example 1 (Synthesis of Aromatic Polycarbonate-based Resin (PC-1) [BPA-Cyclohexyl Diphenolate Copolymer])

82.70 Milliliters of methylene chloride was loaded into a 200-milliliter separable flask including a baffle board, and 2.26 g of cyclohexyl diphenolate obtained in Synthesis Example 1 above was dissolved therein. Subsequently, 111.3 mL of the PCO solution (a) obtained in Synthesis Example 5 above was added to the solution, and then 0.612 g of p-tert-butylphenol (PTBP) was dissolved therein. Next, 13.93 μL of triethylamine (TEA) and 23.42 g of 6.4 mass % aqueous sodium hydroxide (aqueous solution obtained by dissolving 1.50 g of sodium hydroxide in 21.92 mL of pure water) were added to the solution, and the mixture was subjected to a reaction for 20 minutes to provide a polymerization solution (1).

Separately, 3.50 g of sodium hydroxide and 20.35 mg of sodium dithionite were dissolved in 51.16 mL of pure water to provide an aqueous solution. Next, 7.92 g of BPA was dissolved in the aqueous solution to provide a solution (1) of BPA in aqueous sodium hydroxide.

The above-mentioned solution (1) of BPA in aqueous sodium hydroxide was added to the above-mentioned polymerization solution, and the mixture was subjected to a polymerization reaction for 40 minutes. 220 Milliliters of methylene chloride was added for diluting the reaction product, and the mixture was stirred for 5 minutes. After that, the mixture was separated into an organic phase containing a BPA-cyclohexyl diphenolate copolymer, and an aqueous phase containing excess amounts of BPA and sodium hydroxide, and the organic phase was isolated. The solution of the BPA-cyclohexyl diphenolate copolymer in methylene chloride thus obtained was sequentially washed with 0.03 mol/L aqueous sodium hydroxide and 0.2 mol/L hydrochloric acid in amounts of 15 vol % each with respect to the solution. Next, the washed product was repeatedly washed with pure water until an electric conductivity in its aqueous phase after the washing became 5 ρS/cm or less. The washed organic phase was turned into a flake by evaporating its solvent with an evaporator. Thus, a white product was obtained. The product had a viscosity-average molecular weight Mv of 19,600.

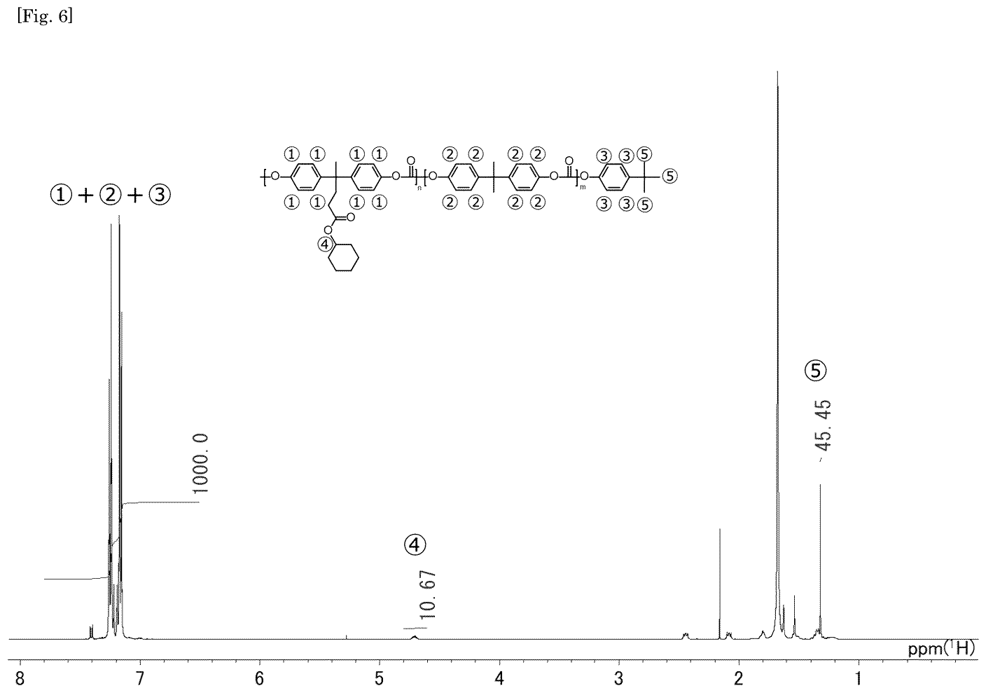

The ¹H-NMR chart of the resultant aromatic polycarbonate-based resin is shown in FIG. 6.

### Production Example 2 (Synthesis of Aromatic Polycarbonate-based Resin (PC-2) [BPA-Cyclopentyl Diphenolate Copolymer])

An aromatic polycarbonate-based resin (PC-2) [BPA-cyclopentyl diphenolate copolymer] was synthesized by performing synthesis through use of cyclopentyl diphenolate obtained in Synthesis Example 2 above instead of cyclohexyl diphenolate in Production Example 1.

Specifically, 62.02 mL of methylene chloride was loaded into a 200-milliliter separable flask including a baffle board, and 5.09 g of cyclopentyl diphenolate obtained in Synthesis Example 2 above was dissolved therein. Subsequently, 87.98 mL of the PCO solution (a) obtained in Synthesis Example 5 above was added to the solution, and then 0.459 g of p-tert-butylphenol (PTBP) was dissolved therein. Next, 4.35 μL of triethylamine (TEA) and 29.28 g of 6.4 mass % aqueous sodium hydroxide (aqueous solution obtained by dissolving 1.87 g of sodium hydroxide in 27.41 mL of pure water) were added to the solution, and the mixture was subjected to a reaction for 20 minutes to provide a polymerization solution (2).

Separately, 1.87 g of sodium hydroxide and 16.74 mg of sodium dithionite were dissolved in 27.41 mL of pure water to provide an aqueous solution. Next, 3.28 g of BPA was dissolved in the aqueous solution to provide a solution (2) of BPA in aqueous sodium hydroxide.

The above-mentioned solution (2) of BPA in aqueous sodium hydroxide was added to the above-mentioned polymerization solution (2), and the mixture was subjected to a polymerization reaction for 40 minutes. 100 Milliliters of methylene chloride was added for diluting the reaction product, and the mixture was stirred for 5 minutes. After that, a solution of the BPA-cyclopentyl diphenolate copolymer in methylene chloride was isolated as an organic phase in the same manner as in Production Example 1. Further, the organic phase was washed, and was then turned into a flake by evaporating its solvent in the same manner as in Production Example 1. Thus, a white product was obtained. The product had a viscosity-average molecular weight My of 15,900.

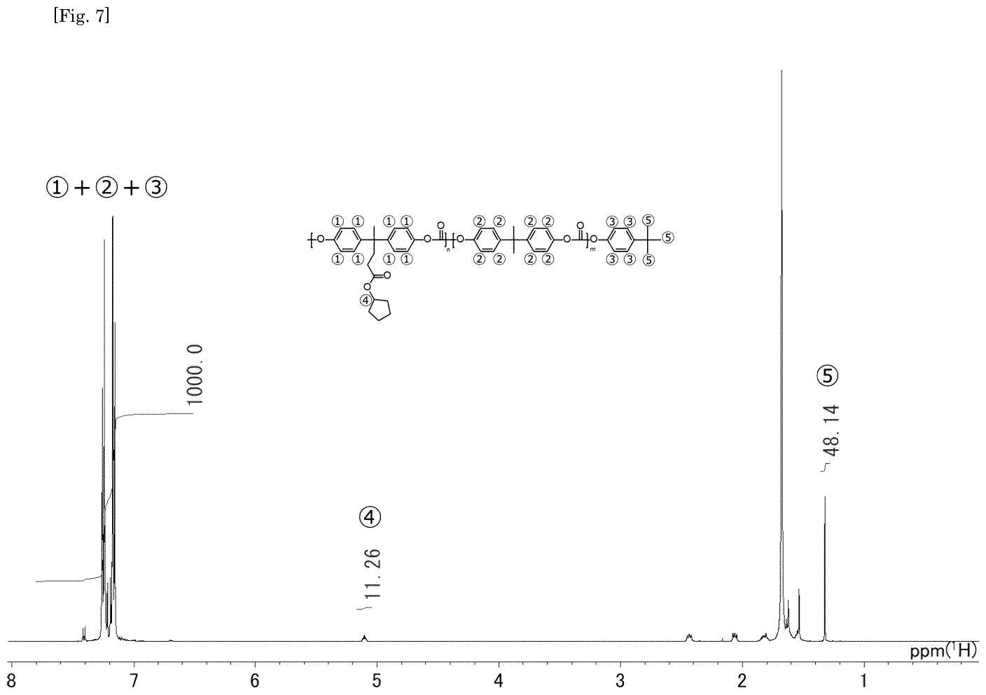

The ¹H-NMR chart of the resultant aromatic polycarbonate-based resin is shown in FIG. 7.

### Production Example 3 (Synthesis of Aromatic Polycarbonate-based Resin (PC-3) [BPA-Methyl Diphenolate Copolymer])

An aromatic polycarbonate-based resin (PC-3) [BPA-methyl diphenolate copolymer] was synthesized by performing synthesis through use of methyl diphenolate obtained in Synthesis Example 3 above instead of cyclohexyl diphenolate in Production Example 1.

Specifically, 62.0 mL of methylene chloride was loaded into a 1-liter separable flask including a baffle board, and 4.50 g of methyl diphenolate obtained in Synthesis Example 3 above was dissolved therein. Subsequently, 88.0 mL of the PCO solution (a) obtained in Synthesis Example 5 above was added to the solution, and then 0.34 g of p-tert-butylphenol (PTBP) was dissolved therein. Next, 17.0 μL of triethylamine (TEA) and 24.3 g of 6.4 mass % aqueous sodium hydroxide (aqueous solution obtained by dissolving 1.55 g of sodium hydroxide in 22.7 mL of pure water) were added to the solution, and the mixture was subjected to a reaction for 10 minutes to provide a polymerization solution (3).

Separately, 2.70 g of sodium hydroxide and 5.98 mg of sodium dithionite were dissolved in 39.5 mL of pure water to provide an aqueous solution. Next, 2.99 g of BPA was dissolved in the aqueous solution to provide a solution (3) of BPA in aqueous sodium hydroxide.

The above-mentioned solution (3) of BPA in aqueous sodium hydroxide was added to the above-mentioned polymerization solution (3), and the mixture was subjected to a polymerization reaction for 50 minutes. 100 Milliliters of methylene chloride was added for diluting the reaction product, and the mixture was stirred for 5 minutes. After that, a solution of the BPA-methyl diphenolate copolymer in methylene chloride was isolated as an organic phase in the same manner as in Production Example 1. Further, the organic phase was washed, and was then turned into a flake by evaporating its solvent in the same manner as in Production Example 1. Thus, a white product was obtained. The product had a viscosity-average molecular weight My of 20,900.

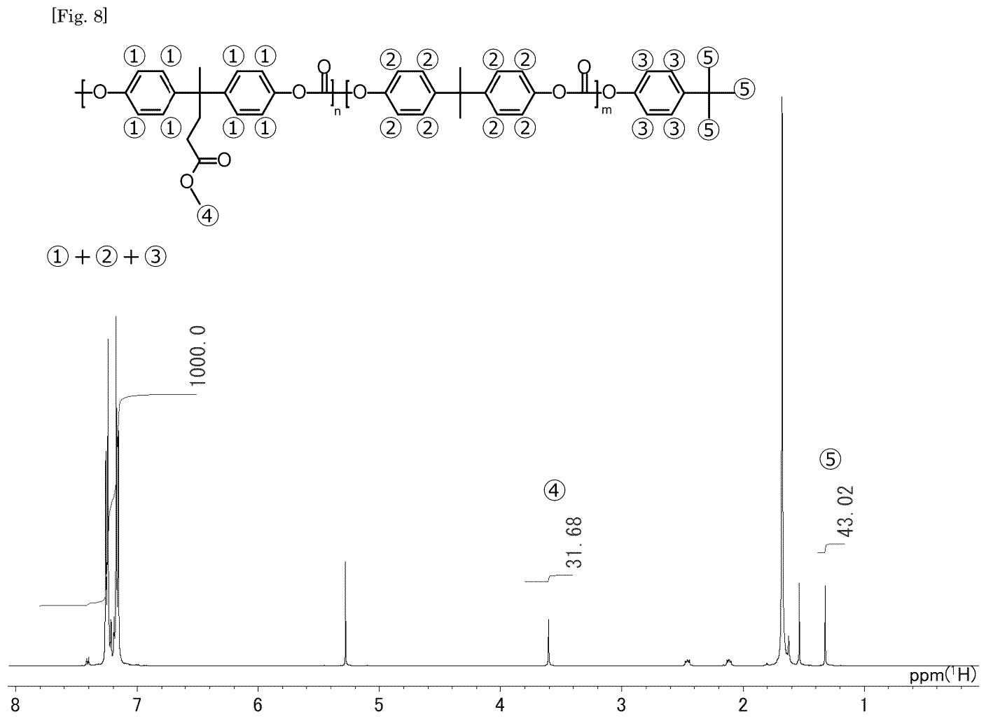

The ¹H-NMR chart of the resultant aromatic polycarbonate-based resin is shown in FIG. 8.

### Production Example 4 (Synthesis of Aromatic Polycarbonate-based Resin (PC-5) [BPA-Cyclohexyl Diphenolate Copolymer])

An aromatic polycarbonate-based resin (PC-5) [BPA-cyclohexyl diphenolate copolymer] was obtained by performing synthesis so that in Production Example 1, the monomer loading ratio “BPA:cyclohexyl diphenolate” became 94:6.

Specifically, 490 mL of methylene chloride, 6.35 g of cyclohexyl diphenolate obtained in Synthesis Example 1 above, and 176 mL of the PCO solution (a) obtained in Synthesis Example 5 above were loaded into a 1-liter separable flask including a baffle board, and then 0.673 g of p-tert-butylphenol (PTBP) was added thereto and dissolved therein. Next, 20.9 μL of triethylamine (TEA) and 35.2 g of 6.4 mass % aqueous sodium hydroxide (aqueous solution obtained by dissolving 2.25 g of sodium hydroxide in 32.9 mL of pure water) were added to the solution, and the mixture was subjected to a reaction for 20 minutes to provide a polymerization solution (4).

Separately, 5.25 g of sodium hydroxide and 31.1 mg of sodium dithionite were dissolved in 76.7 mL of pure water to provide an aqueous solution. Next, 9.18 g of BPA was dissolved in the aqueous solution to provide a solution (4) of BPA in aqueous sodium hydroxide.

48.8 Microliters of triethylamine (TEA) and the above-mentioned solution (4) of BPA in aqueous sodium hydroxide were added to the above-mentioned polymerization solution (4), and the mixture was subjected to a polymerization reaction for 40 minutes. After that, a solution of the BPA-cyclohexyl diphenolate copolymer in methylene chloride was isolated as an organic phase in the same manner as in Production Example 1. Further, the organic phase was washed, and was then turned into a flake by evaporating its solvent in the same manner as in Production Example 1. Thus, a white product was obtained. The product had a viscosity-average molecular weight My of 20,500.

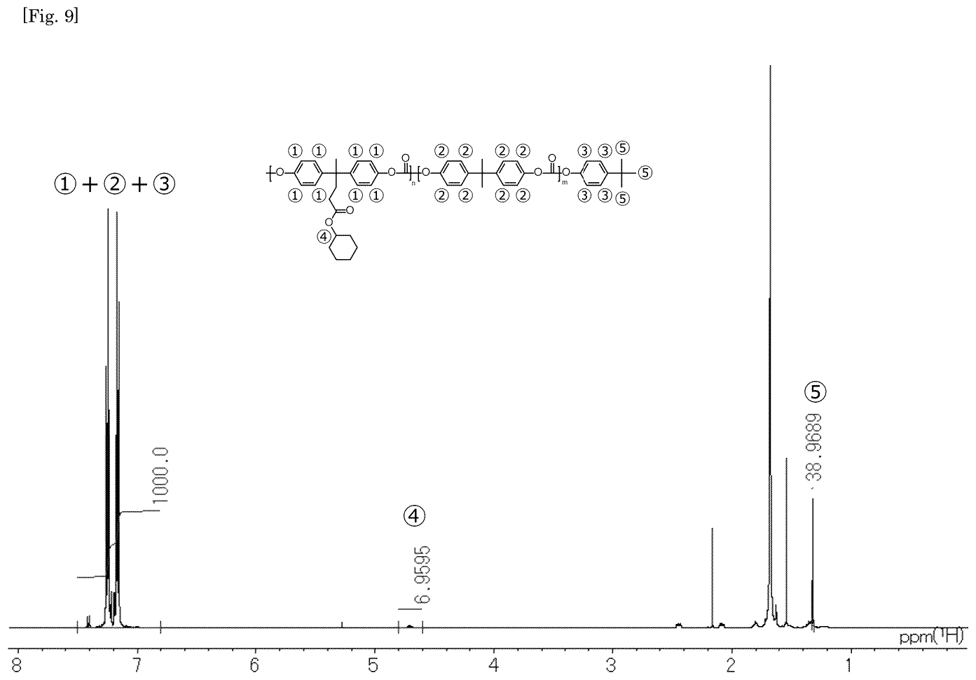

The ¹H-NMR chart of the resultant aromatic polycarbonate-based resin is shown in FIG. 9.

### Production Example 5 (Synthesis of Aromatic Polycarbonate-based Resin (PC-6) [BPA-Cyclohexyl Diphenolate Copolymer])

An aromatic polycarbonate-based resin (PC-6) [BPA-cyclohexyl diphenolate copolymer] was obtained by performing synthesis so that in Production Example 1, the monomer loading ratio “BPA:cyclohexyl diphenolate” became 83:17.

Specifically, 82.7 mL of methylene chloride, 13.7 g of cyclohexyl diphenolate obtained in Synthesis Example 1 above, and 117 mL of the PCO solution (a) obtained in Synthesis Example 5 above were loaded into a 1-liter separable flask including a baffle board, and then 0.612 g of p-tert-butylphenol (PTBP) was added thereto and dissolved therein. Next, 27.9 μL of triethylamine (TEA) and 46.9 g of 6.4 mass % aqueous sodium hydroxide (aqueous solution obtained by dissolving 3.00 g of sodium hydroxide in 43.9 mL of pure water) were added to the solution, and the mixture was subjected to a reaction for 20 minutes to provide a polymerization solution (5).

Separately, 2.00 g of sodium hydroxide and 29.1 mg of sodium dithionite were dissolved in 29.2 mL of pure water to provide an aqueous solution. Next, 0.82 g of BPA was dissolved in the aqueous solution to provide a solution (5) of BPA in aqueous sodium hydroxide.

18.6 Microliters of triethylamine (TEA) and the above-mentioned solution (5) of BPA in aqueous sodium hydroxide were added to the above-mentioned polymerization solution (5), and the mixture was subjected to a polymerization reaction for 40 minutes. After that, a solution of the BPA-cyclohexyl diphenolate copolymer in methylene chloride was isolated as an organic phase in the same manner as in Production Example 1. Further, the organic phase was washed, and was then turned into a flake by evaporating its solvent in the same manner as in Production Example 1. Thus, a white product was obtained. The product had a viscosity-average molecular weight My of 18,000.

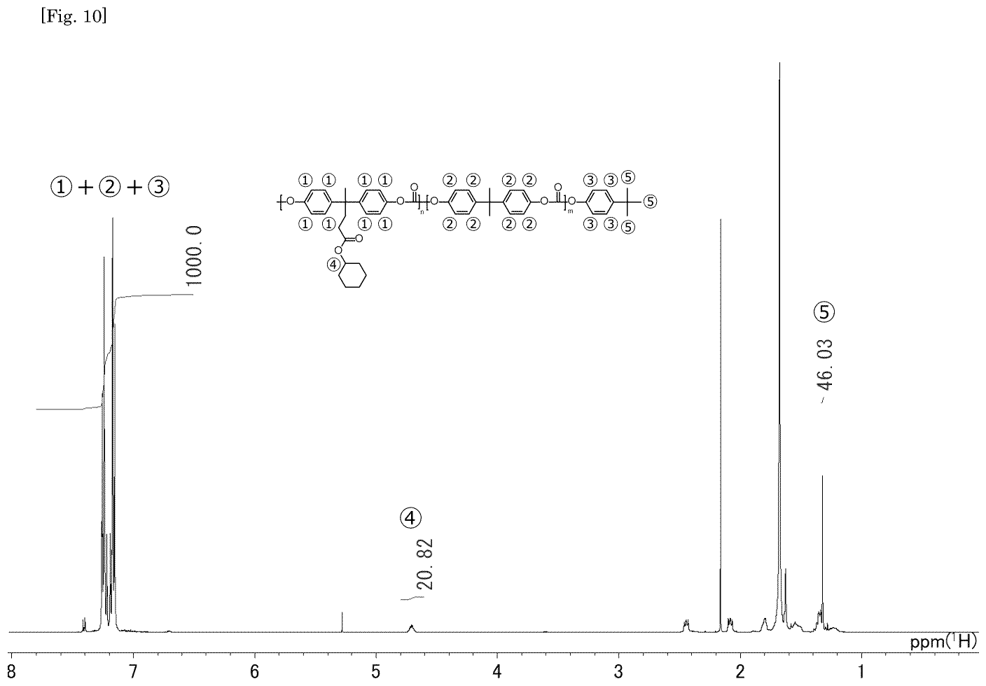

The ¹H-NMR chart of the resultant aromatic polycarbonate-based resin is shown in FIG. 10.

### Production Example 6 (Synthesis of Aromatic Polycarbonate-based Resin (PC-7) [BPA-Cyclohexyl Diphenolate Copolymer])

An aromatic polycarbonate-based resin (PC-7) [BPA-cyclohexyl diphenolate copolymer] was obtained by performing synthesis so that in Production Example 1, the monomer loading ratio “BPA:cyclohexyl diphenolate” became 85:15.

Specifically, 237 mL of methylene chloride, 9.68 g of cyclohexyl diphenolate obtained in Synthesis Example 1 above, 113 ml of the PCO solution (b) obtained in Synthesis Example 6 above, 92.0 μL of triethylamine (TEA), and 36.4 g of 8.1 mass % aqueous sodium hydroxide (aqueous solution obtained by dissolving 2.96 g of sodium hydroxide in 33.4 mL of pure water) were loaded into a 1-liter separable flask including a baffle board, and the materials were dissolved. The solution was subjected to a reaction for 20 minutes to provide a polymerization solution (6).

Separately, 5.10 g of sodium hydroxide and 17.0 mg of sodium dithionite were dissolved in 75.1 mL of pure water to provide an aqueous solution. Next, 8.30 g of BPA was dissolved in the aqueous solution to provide a solution (6) of BPA in aqueous sodium hydroxide.

6.00 Milliliters of a methylene chloride solution having dissolved therein 0.80 g of p-tert-butylphenol (PTBP) and the above-mentioned solution (6) of BPA in aqueous sodium hydroxide were added to the above-mentioned polymerization solution (6), and the mixture was subjected to a polymerization reaction for 40 minutes. 373 Milliliters of methylene chloride was added for diluting the reaction product, and the mixture was stirred for 5 minutes. After that, a solution of the BPA-cyclohexyl diphenolate copolymer in methylene chloride was isolated as an organic phase in the same manner as in Production Example 1. Further, the organic phase was washed, and was then turned into a flake by evaporating its solvent in the same manner as in Production Example 1. Thus, a white product was obtained. The product had a viscosity-average molecular weight My of 23,300.

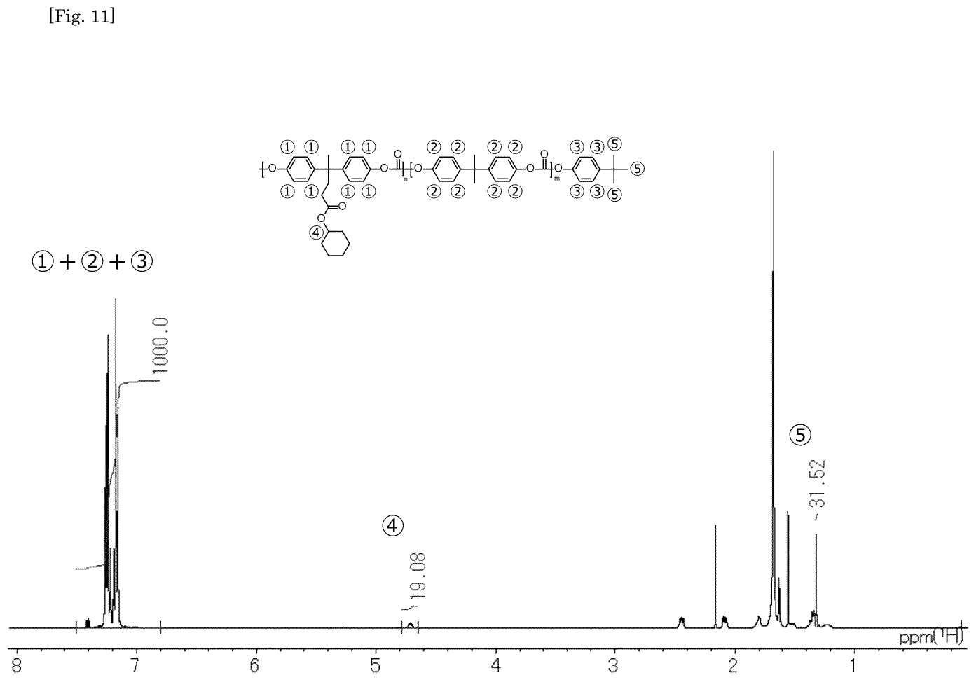

The ¹H-NMR chart of the resultant aromatic polycarbonate-based resin is shown in FIG. 11.

### Production Example 7 (Synthesis of Aromatic Polycarbonate-based Resin (PC-8) [BPA-Cyclohexyl Diphenolate Copolymer])

An aromatic polycarbonate-based resin (PC-8) [BPA-cyclohexyl diphenolate copolymer] was obtained by performing synthesis so that in Production Example 1, the monomer loading ratio “BPA:cyclohexyl diphenolate” became 89:11.

Specifically, 237 mL of methylene chloride, 6.83 g of cyclohexyl diphenolate obtained in Synthesis Example 1 above, 113 ml of the PCO solution (b) obtained in Synthesis Example 6 above, 92.0 μL of triethylamine (TEA), and 36.4 g of 8.1 mass % aqueous sodium hydroxide (aqueous solution obtained by dissolving 2.95 g of sodium hydroxide in 33.4 mL of pure water) were loaded into a 1-liter separable flask including a baffle board, and the materials were dissolved. The solution was subjected to a reaction for 20 minutes to provide a polymerization solution (7).

Separately, 5.10 g of sodium hydroxide and 17.0 mg of sodium dithionite were dissolved in 75.0 mL of pure water to provide an aqueous solution. Next, 8.31 g of BPA was dissolved in the aqueous solution to provide a solution (7) of BPA in aqueous sodium hydroxide.

6.00 Milliliters of a methylene chloride solution having dissolved therein 0.81 g of p-tert-butylphenol (PTBP) and the above-mentioned solution (7) of BPA in aqueous sodium hydroxide were added to the above-mentioned polymerization solution (7), and the mixture was subjected to a polymerization reaction for 40 minutes. 331 Milliliters of methylene chloride was added for diluting the reaction product, and the mixture was stirred for 5 minutes. After that, a solution of the BPA-cyclohexyl diphenolate copolymer in methylene chloride was isolated as an organic phase in the same manner as in Production Example 1. Further, the organic phase was washed, and was then turned into a flake by evaporating its solvent in the same manner as in Production Example 1. Thus, a white product was obtained. The product had a viscosity-average molecular weight My of 22,000.

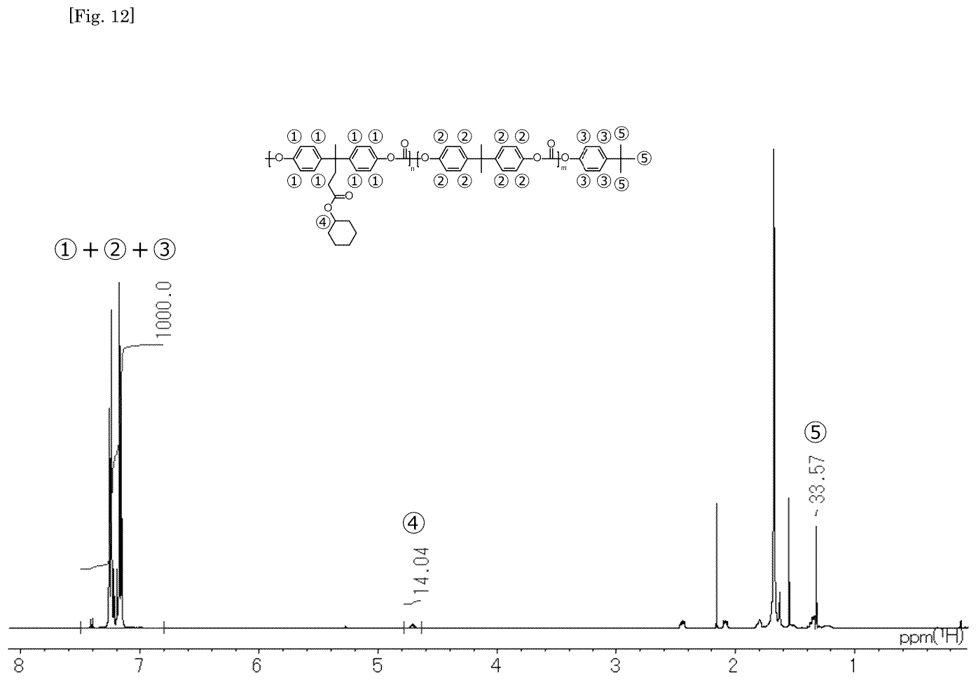

The ¹H-NMR chart of the resultant aromatic polycarbonate-based resin is shown in FIG. 12.

### Production Example 8 (Synthesis of Aromatic Polycarbonate-based Resin (PC-9) [BPA-Cyclopentyl Diphenolate Copolymer])

An aromatic polycarbonate-based resin (PC-9) [BPA-cyclopentyl diphenolate copolymer] was obtained by performing synthesis so that in Production Example 2, the monomer loading ratio “BPA:cyclopentyl diphenolate” became 94:6.

Specifically, 491 mL of methylene chloride, 6.11 g of cyclopentyl diphenolate obtained in Synthesis Example 2 above, and 176 mL of the PCO solution (a) obtained in Synthesis Example 5 above were loaded into a 1-liter separable flask including a baffle board, and then 0.673 g of p-tert-butylphenol (PTBP) was added thereto and dissolved therein. Next, 20.9 μL of triethylamine (TEA) and 35.2 g of 6.4 mass % aqueous sodium hydroxide (aqueous solution obtained by dissolving 2.25 g of sodium hydroxide in 32.9 mL of pure water) were added to the solution, and the mixture was subjected to a reaction for 20 minutes to provide a polymerization solution (8).

Separately, 5.25 g of sodium hydroxide and 31.1 mg of sodium dithionite were dissolved in 76.7 mL of pure water to provide an aqueous solution. Next, 9.18 g of BPA was dissolved in the aqueous solution to provide a solution (8) of BPA in aqueous sodium hydroxide.

48.8 Microliters of triethylamine (TEA) and the above-mentioned solution (8) of BPA in aqueous sodium hydroxide were added to the above-mentioned polymerization solution (8), and the mixture was subjected to a polymerization reaction for 40 minutes. After that, a solution of the BPA-cyclopentyl diphenolate copolymer in methylene chloride was isolated as an organic phase in the same manner as in Production Example 1. Further, the organic phase was washed, and was then turned into a flake by evaporating its solvent in the same manner as in Production Example 1. Thus, a white product was obtained. The product had a viscosity-average molecular weight My of 21,200.

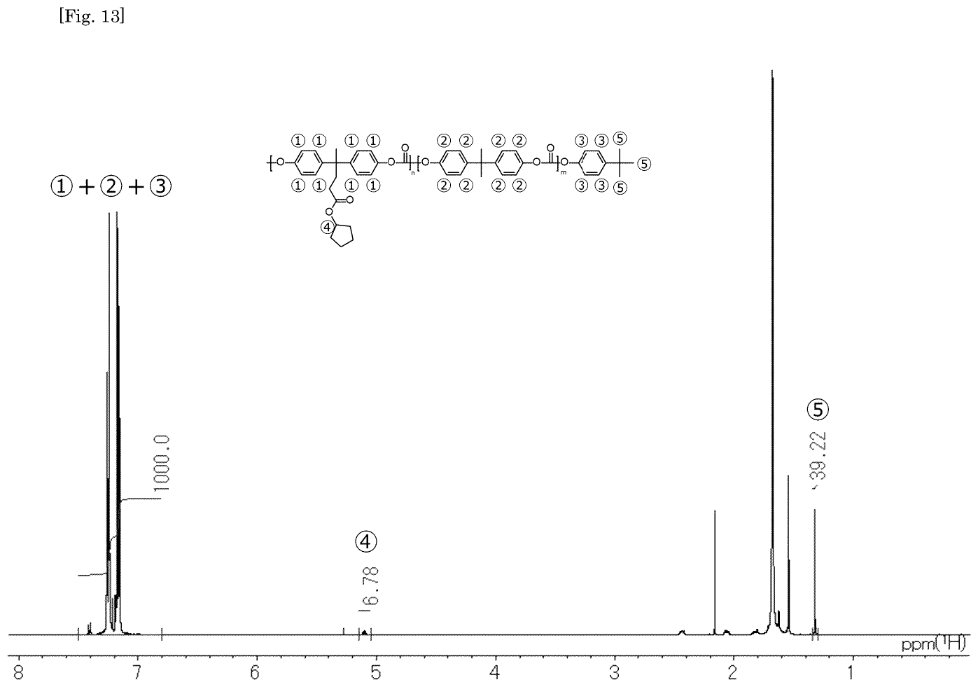

The ¹H-NMR chart of the resultant aromatic polycarbonate-based resin is shown in FIG. 13.

### Production Example 9 (Synthesis of Aromatic Polycarbonate-based Resin (PC-10) [BPA-Cyclopentyl Diphenolate Copolymer])

An aromatic polycarbonate-based resin (PC-10) [BPA-cyclopentyl diphenolate copolymer] was obtained by performing synthesis so that in Production Example 2, the monomer loading ratio “BPA:cyclopentyl diphenolate” became 96:4.

Specifically, 41.4 mL of methylene chloride, 1.51 g of cyclopentyl diphenolate obtained in Synthesis Example 2 above, and 58.6 mL of the PCO solution (a) obtained in Synthesis Example 5 above were loaded into a 200-milliliter separable flask including a baffle board, and then 0.100 g of p-tert-butylphenol (PTBP) was added thereto and dissolved therein. Next, 1.16 μL of triethylamine (TEA) and 5.2 g of 6.4 mass % aqueous sodium hydroxide (aqueous solution obtained by dissolving 0.33 g of sodium hydroxide in 4.87 mL of pure water) were added to the solution, and the mixture was subjected to a reaction for 20 minutes to provide a polymerization solution (9).

Separately, 2.50 g of sodium hydroxide and 7.76 mg of sodium dithionite were dissolved in 36.5 mL of pure water to provide an aqueous solution. Next, 3.88 g of BPA was dissolved in the aqueous solution to provide a solution (9) of BPA in aqueous sodium hydroxide.

4.64 Microliters of triethylamine (TEA) and the above-mentioned solution (9) of BPA in aqueous sodium hydroxide were added to the above-mentioned polymerization solution (9), and the mixture was subjected to a polymerization reaction for 40 minutes. 100 Milliliters of methylene chloride was added for diluting the reaction product, and the mixture was stirred for 5 minutes. After that, a solution of the BPA-cyclopentyl diphenolate copolymer in methylene chloride was isolated as an organic phase in the same manner as in Production Example 1. Further, the organic phase was washed, and was then turned into a flake by evaporating its solvent in the same manner as in Production Example 1. Thus, a white product was obtained. The product had a viscosity-average molecular weight My of 25,300.

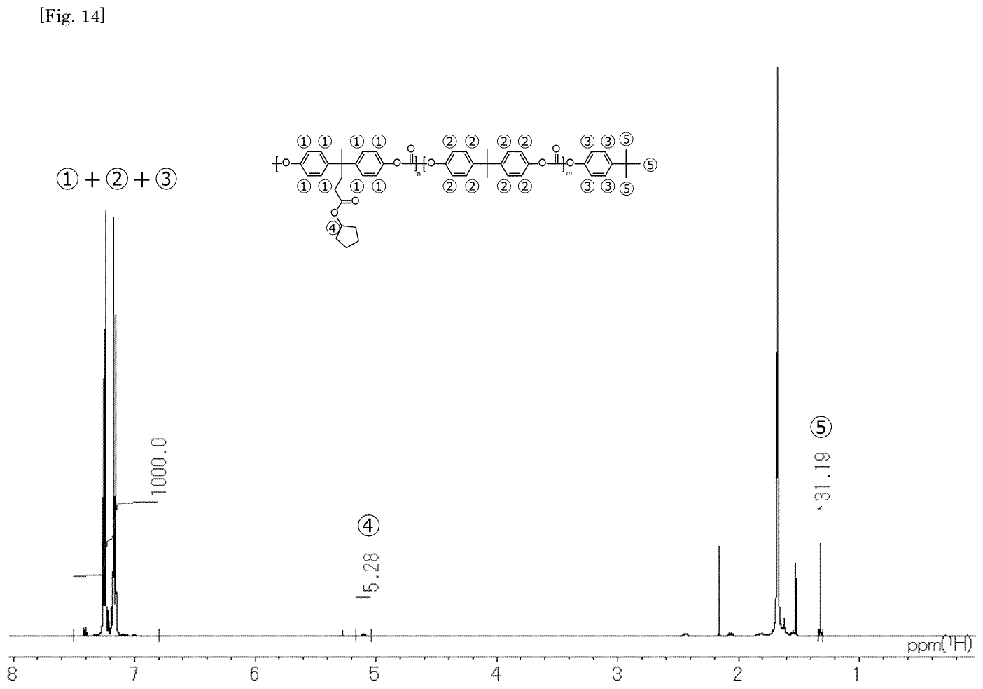

The ¹H-NMR chart of the resultant aromatic polycarbonate-based resin is shown in FIG. 14.

### Production Example 10 (Synthesis of Aromatic Polycarbonate-Based Resin (PC-11) [BPA-Cyclopentyl Diphenolate Copolymer])

An aromatic polycarbonate-based resin (PC-11) [BPA-cyclopentyl diphenolate copolymer] was obtained by performing synthesis so that in Production Example 2, the monomer loading ratio “BPA:cyclopentyl diphenolate” became 89:11.

Specifically, 154 mL of methylene chloride, 8.84 g of cyclopentyl diphenolate obtained in Synthesis Example 2 above, 146 ml of the PCO solution (b) obtained in Synthesis Example 6 above, 118 μL of triethylamine (TEA), and 27.4 g of 8.0 mass % aqueous sodium hydroxide (aqueous solution obtained by dissolving 2.20 g of sodium hydroxide in 25.2 mL of pure water) were loaded into a 1-liter separable flask including a baffle board, and the materials were dissolved. The solution was subjected to a reaction for 20 minutes to provide a polymerization solution (10).

Separately, 6.60 g of sodium hydroxide and 21.0 mg of sodium dithionite were dissolved in 96.0 mL of pure water to provide an aqueous solution. Next, 10.6 g of BPA was dissolved in the aqueous solution to provide a solution (10) of BPA in aqueous sodium hydroxide.

8.00 Milliliters of a methylene chloride solution having dissolved therein 1.10 g of p-tert-butylphenol (PTBP) and the above-mentioned solution (10) of BPA in aqueous sodium hydroxide were added to the above-mentioned polymerization solution (10), and the mixture was subjected to a polymerization reaction for 40 minutes. 322 Milliliters of methylene chloride was added for diluting the reaction product, and the mixture was stirred for 5 minutes. After that, a solution of the BPA-cyclopentyl diphenolate copolymer in methylene chloride was isolated as an organic phase in the same manner as in Production Example 1. Further, the organic phase was washed, and was then turned into a flake by evaporating its solvent in the same manner as in Production Example 1. Thus, a white product was obtained. The product had a viscosity-average molecular weight My of 25,100.

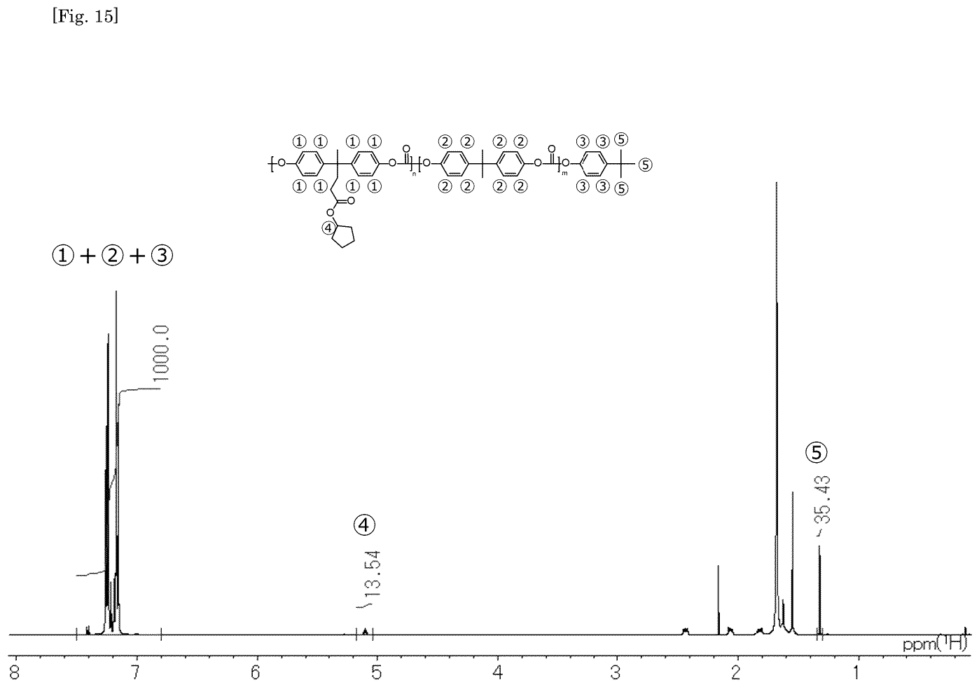

The ¹H-NMR chart of the resultant aromatic polycarbonate-based resin is shown in FIG. 15.

### Production Example 11 (Synthesis of Aromatic Polycarbonate-based Resin (PC-12) [BPA-Cyclohexyl 2,2-Bis(4-hydroxyphenyl)propanoate Copolymer])

An aromatic polycarbonate-based resin (PC-12) [BPA-cyclohexyl 2,2-bis(4-hydroxyphenyl)propanoate copolymer] was synthesized by performing synthesis through use of cyclohexyl 2,2-bis(4-hydroxyphenyl)propanoate obtained in Synthesis Example 4 above instead of cyclohexyl diphenolate in Production Example 1 so that the monomer loading ratio “BPA:cyclohexyl 2,2-bis(4-hydroxyphenyl)propanoate” became 92:8.

Specifically, first, 6.18 g of cyclohexyl 2,2-bis(4-hydroxyphenyl)propanoate obtained in Synthesis Example 4 above was dissolved in 27.4 g of 8.0 mass % aqueous sodium hydroxide (aqueous solution obtained by dissolving 2.20 g of sodium hydroxide in 25.2 mL of pure water) to provide an aqueous solution of cyclohexyl 2,2-bis(4-hydroxyphenyl)propanoate.

The aqueous solution of cyclohexyl 2,2-bis(4-hydroxyphenyl)propanoate, 146 ml of the PCO solution (b) obtained in Synthesis Example 6 above, 154 mL of methylene chloride, and 118 μL of triethylamine (TEA) were loaded into a 1-liter separable flask including a baffle board, and the mixture was subjected to a reaction for 20 minutes to provide a polymerization solution (11).

Separately, 6.60 g of sodium hydroxide and 21.0 mg of sodium dithionite were dissolved in 96.1 mL of pure water to provide an aqueous solution. Next, 10.6 g of BPA was dissolved in the aqueous solution to provide a solution (11) of BPA in aqueous sodium hydroxide.

8.00 Milliliters of a methylene chloride solution having dissolved therein 1.10 g of p-tert-butylphenol (PTBP) and the above-mentioned solution (11) of BPA in aqueous sodium hydroxide were added to the above-mentioned polymerization solution (11), and the mixture was subjected to a polymerization reaction for 40 minutes. 373 Milliliters of methylene chloride was added for diluting the reaction product, and the mixture was stirred for 5 minutes. After that, a solution of the BPA-cyclohexyl 2,2-bis(4-hydroxyphenyl)propanoate copolymer in methylene chloride was isolated as an organic phase in the same manner as in Production Example 1. Further, the organic phase was washed, and was then turned into a flake by evaporating its solvent in the same manner as in Production Example 1. Thus, a white product was obtained. The product had a viscosity-average molecular weight My of 21,900.

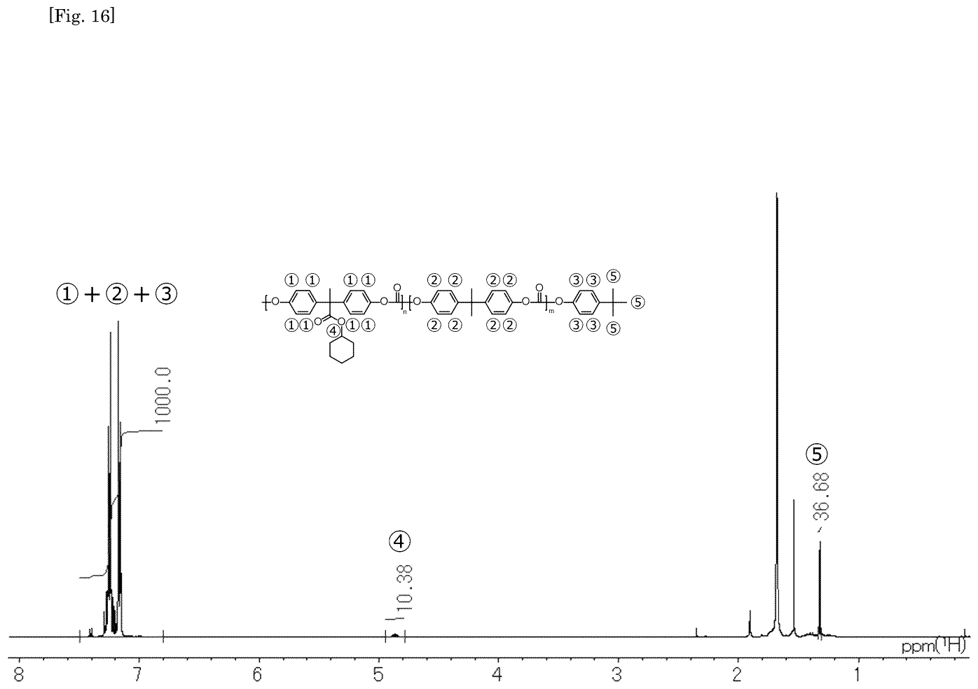

The ¹H-NMR chart of the resultant aromatic polycarbonate-based resin is shown in FIG. 16.

### Production Example 12 (Synthesis of Aromatic Polycarbonate-based Resin (PC-13) [BPA-Cyclohexyl 2,2-Bis(4-hydroxyphenyl)propanoate Copolymer])

An aromatic polycarbonate-based resin (PC-13) [BPA-cyclohexyl 2,2-bis(4-hydroxyphenyl)propanoate copolymer] was obtained by performing synthesis so that in Production Example 2, the monomer loading ratio “BPA:cyclohexyl 2,2-bis(4-hydroxyphenyl)propanoate” became 84:16.

Specifically, first, 11.1 g of cyclohexyl 2,2-bis(4-hydroxyphenyl)propanoate obtained in Synthesis Example 4 above was dissolved in 41.4 g of 8.0 mass % aqueous sodium hydroxide (aqueous solution obtained by dissolving 3.30 g of sodium hydroxide in 38.1 mL of pure water) to provide an aqueous solution of cyclohexyl 2,2-bis(4-hydroxyphenyl)propanoate.

The aqueous solution of cyclohexyl 2,2-bis(4-hydroxyphenyl)propanoate, 137 ml of the PCO solution (b) obtained in Synthesis Example 6 above, and 105 μL of triethylamine (TEA) were loaded into a 1-liter separable flask including a baffle board, and the mixture was subjected to a reaction for 20 minutes to provide a polymerization solution (12).

Separately, 5.90 g of sodium hydroxide and 19.0 mg of sodium dithionite were dissolved in 86.0 mL of pure water to provide an aqueous solution. Next, 9.40 g of BPA was dissolved in the aqueous solution to provide a solution (12) of BPA in aqueous sodium hydroxide.

6.00 Milliliters of a methylene chloride solution having dissolved therein 0.86 g of p-tert-butylphenol (PTBP) and the above-mentioned solution (12) of BPA in aqueous sodium hydroxide were added to the above-mentioned polymerization solution (12), and the mixture was subjected to a polymerization reaction for 40 minutes. 36.0 Milliliters of methylene chloride was added for diluting the reaction product, and the mixture was stirred for 5 minutes. After that, a solution of the BPA-cyclohexyl 2,2-bis(4-hydroxyphenyl)propanoate copolymer in methylene chloride was isolated as an organic phase in the same manner as in Production Example 1. Further, the organic phase was washed, and was then turned into a flake by evaporating its solvent in the same manner as in Production Example 1. Thus, a white product was obtained. The product had a viscosity-average molecular weight My of 23,500.

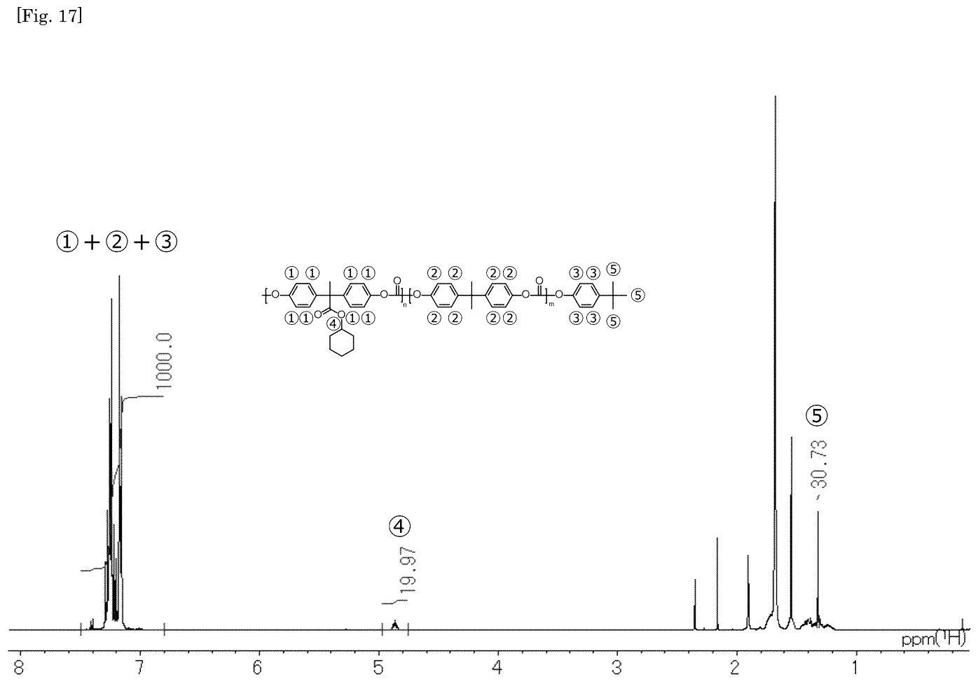

The ¹H-NMR chart of the resultant aromatic polycarbonate-based resin is shown in FIG. 17.

### Production Example 13 (Synthesis of Aromatic Polycarbonate-based Resin (PC-14) [BPA-Methyl Diphenolate Copolymer])

An aromatic polycarbonate-based resin (PC-14) [BPA-methyl diphenolate copolymer] was obtained by performing synthesis so that in Production Example 3, the monomer loading ratio “BPA:methyl diphenolate” became 96:4.

491 Milliliters of methylene chloride, 5.18 g of methyl diphenolate obtained in Synthesis Example 3 above, and 176 mL of the PCO solution (a) obtained in Synthesis Example 5 above were loaded into a 1-liter separable flask including a baffle board, and then 0.673 g of p-tert-butylphenol (PTBP) was added thereto and dissolved therein. Next, 20.9 μL of triethylamine (TEA) and 35.2 g of 6.4 mass % aqueous sodium hydroxide (aqueous solution obtained by dissolving 2.25 g of sodium hydroxide in 32.9 mL of pure water) were added to the solution, and the mixture was subjected to a reaction for 20 minutes to provide a polymerization solution (13).

Separately, 5.25 g of sodium hydroxide and 31.1 mg of sodium dithionite were dissolved in 76.7 mL of pure water to provide an aqueous solution. Next, 9.18 g of BPA was dissolved in the aqueous solution to provide a solution (13) of BPA in aqueous sodium hydroxide.

48.8 Microliters of triethylamine (TEA) and the above-mentioned solution (13) of BPA in aqueous sodium hydroxide were added to the above-mentioned polymerization solution (13), and the mixture was subjected to a polymerization reaction for 40 minutes. After that, a solution of the BPA-methyl diphenolate copolymer in methylene chloride was isolated as an organic phase in the same manner as in Production Example 1. Further, the organic phase was washed, and was then turned into a flake by evaporating its solvent in the same manner as in Production Example 1. Thus, a white product was obtained. The product had a viscosity-average molecular weight My of 22,100.

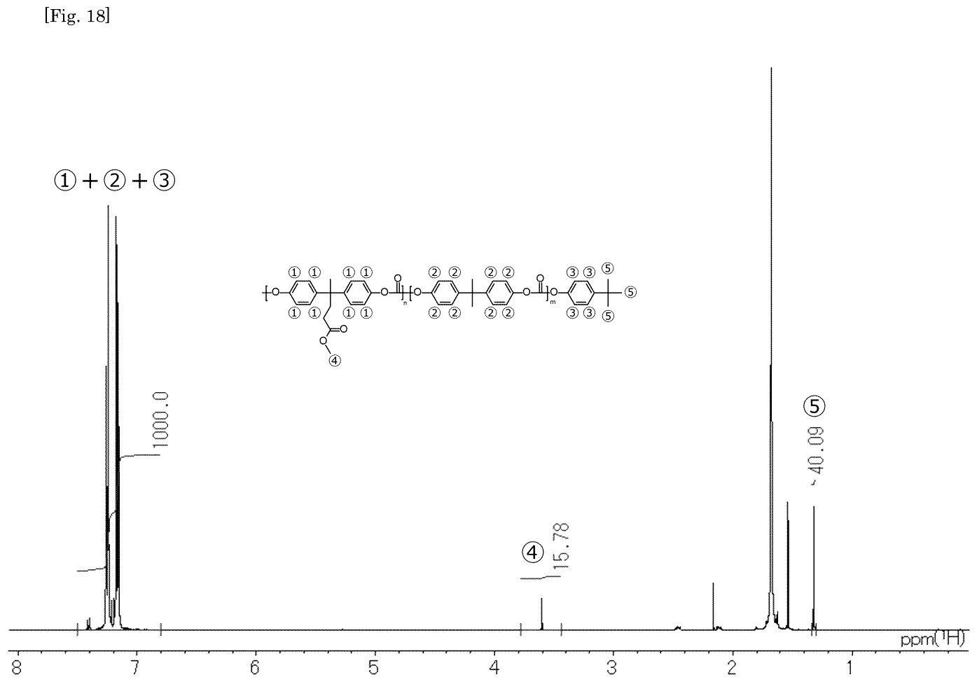

The ¹H-NMR chart of the resultant aromatic polycarbonate-based resin is shown in FIG. 18.

### Examples 1 to 11 and Comparative Examples 1 to 3

### (1) Evaluation of Scratch Hardness (Pencil Method)

The aromatic polycarbonate-based resins (PC-1) to (PC-3) and (PC-5) to (PC-14) obtained in Production Examples 1 to 13, and “TARFLON FN1900” (product name, manufactured by Idemitsu Kosan Co., Ltd., aromatic polycarbonate-based resin formed of BPA, viscosity-average molecular weight Mv: 19,100) serving as an aromatic polycarbonate-based resin (PC-4) in Comparative Example 2 were each subjected to injection molding with an injection molding machine (“Mini Jet Pro” manufactured by Thermo Fisher Scientific, Inc.) under the conditions of a cylinder temperature of 290° C. and a mold temperature of 90° C. to produce a disc-shaped molded body (having a diameter of 30 mm and a thickness of 1.5 mm).

A line was drawn on the surface of the molded body on the basis of JIS K5600-5-4:1999 with a pencil under a state in which a load of 750 g was applied to the pencil while an angle of 45° was kept. The presence or absence of a scratch on the surface was visually inspected, and the scratch hardness (pencil method) of the surface was evaluated. A pencil hardness determined by the scratch hardness (pencil method) is a pencil hardness on any one of the following 14 stages: 6B to B, HB, F, and H to 6H.

The results are shown in Table 1.

### (2) Measurement of Total Light Transmittance

The total light transmittance [%] of each of the molded bodies produced in the above-mentioned section (1) when the molded body had a thickness of 1.5 mm was measured with a haze meter NDH 5000 (manufactured by Nippon Denshoku Industries Co., Ltd.) in conformity with ASTM D1003-21.

The results are shown in Table 1 and Table 2.

### (3) Notched Izod Impact Strength

Notched Izod impact strengths [kJ/m²] were measured by using the aromatic polycarbonate-based resins (PC-5, PC-9, PC-10, and PC-14) obtained in Production Examples 4, 8, 9, and 13 above, and the above-mentioned “TARFLON FN1900” serving as the aromatic polycarbonate-based resin (PC-4).

Specifically, the resins were each subjected to injection molding with an injection molding machine (“Mini Jet Pro” manufactured by Thermo Fisher Scientific, Inc.) under the conditions of a cylinder temperature of from 270° C. to 290° C. and a mold temperature of from 80° C. to 100° C. to produce a strip-shaped molded body (having a length of 60 mm, a width of 40 mm, and a thickness of 4 mm). A notch (r=0.25 mm±0.05 mm) was made at a position of the molded body corresponding to a length of 30 mm by postprocessing to provide a notched test piece. A notched Izod impact strength at 23° C. was measured so that a pendulum hammer was brought into abutment with a portion above the notch portion of the test piece by 22 mm.

In each of Table 1 and Table 2, a case in which the notched Izod impact strength was 9 kJ/m² or more was indicated by A.

### (4) Indentation Hardness

The indentation hardness [MPa] of each of the molded bodies of Example 2, Example 3, Example 7, Example 8, Comparative Example 2, and Comparative Example 3 produced in the above-mentioned section (1) at 28° C. was measured with an ultramicro indentation hardness tester ENT-1100a (manufactured by Elionix Inc.) by a nanoindentation test in conformity with ISO 14577-1:2015. A Vickers (square pyramid) indenter was used as an indenter, and a test load was set to 96 mN.

The results are shown in Table 1 and Table 2.

TABLE 1

| | | | | Comparative | |
|---|---|---|---|---|---|
| | | Example | | Example | |
| | | 1 | 2 | 1 | 2 |
| Aromatic polycarbonate- | | PC-1 | PC-2 | PC-3 | PC-4 |
| based resin | | | | | |
| Formula | Kind | BPA | BPA | BPA | BPA |
| (I) | Content [mol %] | 91 | 91 | 91 | 100 |
| Formula | Kind | CyHex | CyPen | Me | |
| (II) | | (1) | | | |
| | Content [mol %] | 9 | 9 | 9 | 0 |
| | Alkyl chain length (n) | 2 | 2 | 2 | 2 |

| | | | | |
|---|---|---|---|---|
| Scratch hardness | F | HB | HB | 2B |
| Total light transmittance [%] | 89 | 89 | 88 | 89 |
| Notched Izod impact strength | — | — | — | A |
| Indentation hardness [MPa] | — | 163 | — | 149 |

TABLE 2

| | | | | | | | | | | | Comparative |
|---|---|---|---|---|---|---|---|---|---|---|---|
| | | Example | | | | | | | | | Example |
| | | 3 | 4 | 5 | 6 | 7 | 8 | 9 | 10 | 11 | 3 |
| Aromatic | | PC-5 | PC-6 | PC-7 | PC-8 | PC-9 | PC-10 | PC-11 | PC-12 | PC-13 | PC-14 |
| polycarbonate- | | | | | | | | | | | |
| based resin | | | | | | | | | | | |
| Formula | Kind | BPA | BPA | BPA | BPA | BPA | BPA | BPA | BPA | BPA | BPA |
| (I) | Content | 94 | 83 | 85 | 89 | 94 | 96 | 89 | 92 | 84 | 96 |
| | [mol %] | | | | | | | | | | |
| Formula | Kind | CyHex (1) | CyHex (1) | CyHex (1) | CyHex (1) | CyPen | CyPen | CyPen | CyHex (2) | CyHex (2) | Me |
| (II) | Content | 6 | 17 | 15 | 11 | 6 | 4 | 11 | 8 | 16 | 4 |
| | [mol | | | | | | | | | | |
| | Alkyl | 2 | 2 | 2 | 2 | 2 | 2 | 2 | 0 | 0 | 2 |
| | chain | | | | | | | | | | |
| | length | | | | | | | | | | |
| | (n) | | | | | | | | | | |

| | | | | | | | | | | |
|---|---|---|---|---|---|---|---|---|---|---|
| Scratch | F | F | F | F | HB | HB | F | F | F | HB |
| hardness | | | | | | | | | | |
| Total light | 89 | 89 | 90 | 90 | 89 | 89 | 89 | 89 | 89 | 88 |
| transmittance | | | | | | | | | | |
| [%] | | | | | | | | | | |
| Notched Izod | A | — | — | — | A | A | — | — | — | A |
| impact | | | | | | | | | | |
| strength | | | | | | | | | | |
| Indentation | 192 | — | — | — | 155 | 155 | — | — | — | 154 |
| hardness | | | | | | | | | | |
| [MPa] | | | | | | | | | | |

Abbreviations in the tables are as described below.
BPA: bisphenol A
CyHex(1): cyclohexyl diphenolate
CyPen: cyclopentyl diphenolate
Me: methyl diphenolate
CyHex(2): cyclohexyl 2,2-bis(4-hydroxyphenyl)propanoate

In addition, the symbol “−” in each of the tables means that no measurement was performed.

The invention claimed is:

1. An aromatic polycarbonate-based resin, comprising a repeating unit represented by the following formula (II): 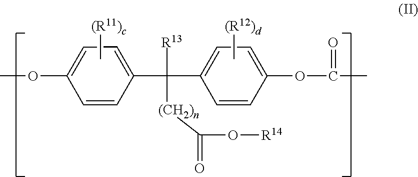
wherein in the formula (II),
R¹¹ and R¹² each independently represent a halogen atom, or a group selected from the group consisting of: an alkyl group having 1 to 18 carbon atoms; an alkoxy group having 1 to 18 carbon atoms; a cycloalkyl group having 3 to 20 carbon atoms; a cycloalkoxy group having 6 to 20 carbon atoms; an alkenyl group having 2 to 10 carbon atoms; an aryl group having 6 to 14 carbon atoms; an aryloxy group having 6 to 14 carbon atoms; an aralkyl group having 7 to 20 carbon atoms; an aralkyloxy group having 7 to 20 carbon atoms; a nitro group; an aldehyde group; a cyano group; and a carboxyl group,
R¹³ represents a hydrogen atom, or a group selected from the group consisting of: an alkyl group having 1 to 5 carbon atoms; a cycloalkyl group having 3 to 20 carbon atoms; a cycloalkoxy group having 3 to 20 carbon atoms; an alkenyl group having 2 to 10 carbon atoms; and an aryl group having 6 to 14 carbon atoms,
R¹⁴ represents a saturated or unsaturated alicyclic group having 3 to 20 carbon atoms, or a 3- to 20-membered saturated or unsaturated heterocyclic group,
“c” and “d” each independently represent an integer of from 0 to 4, and
“n” represents an integer of from 0 to 20.

2. The aromatic polycarbonate-based resin according to claim 1, further comprising a repeating unit represented by the following formula (I), wherein a molar ratio ((I):(II)) between the repeating unit represented by the formula (I) and the repeating unit represented by the formula (II) is from 0:100 to 99.5:0.5: 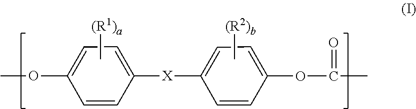
wherein in the formula (I),
R¹ and R² each independently represent a halogen atom, or a group selected from the group consisting of: an alkyl group having 1 to 18 carbon atoms; an alkoxy group having 1 to 18 carbon atoms; a cycloalkyl group having 6 to 20 carbon atoms; a cycloalkoxy group having 6 to 20 carbon atoms; an alkenyl group having 2 to 10 carbon atoms; an aryl group having 6 to 14 carbon atoms; an aryloxy group having 6 to 14 carbon atoms; an aralkyl group having 7 to 20 carbon atoms; an aralkyloxy group having 7 to 20 carbon atoms; a nitro group; an aldehyde group; a cyano group; and a carboxyl group,
X represents a single bond, an alkylene group having 1 to 8 carbon atoms, an alkylidene group having 2 to 8 carbon atoms, a cycloalkylene group having 5 to 15 carbon atoms, a cycloalkylidene group having 5 to 15 carbon atoms, an aralkyl group having 7 to 20 carbon atoms, —S—, —SO—, —SO₂—, —O—, or —CO—, and
“a” and “b” each independently represent an integer of from 0 to 4.

3. The aromatic polycarbonate-based resin according to claim 2, wherein the molar ratio ((I):(II)) between the repeating unit represented by the formula (I) and the repeating unit represented by the formula (II) is from 0.5:99.5 to 99.5:0.5.

4. The aromatic polycarbonate-based resin according to claim 2, wherein the molar ratio ((I):(II)) between the repeating unit represented by the formula (I) and the repeating unit represented by the formula (II) is from 60:40 to 99.5:0.5.

5. The aromatic polycarbonate-based resin according to claim 1, wherein R¹⁴ represents a saturated or unsaturated alicyclic group having 3 to 12 carbon atoms, or a 3- to 12-membered saturated or unsaturated heterocyclic group.

6. The aromatic polycarbonate-based resin according to claim 1, wherein R¹⁴ represents a cyclopentyl group or a cyclohexyl group, and “n” represents 2.

7. The aromatic polycarbonate-based resin according to claim 1, wherein the aromatic polycarbonate-based resin has a viscosity-average molecular weight of from 10,000 to 100,000.

8. The aromatic polycarbonate-based resin according to claim 1, wherein the aromatic polycarbonate-based resin has a scratch hardness of F or more, which is evaluated in conformity with JIS K5600-5-4.

9. The aromatic polycarbonate-based resin according to claim 1, wherein the aromatic polycarbonate-based resin has a total light transmittance of 87% or more when molded into a thickness of 1.5 mm.

10. A dihydric phenol-based compound, which is represented by the following formula (ii): 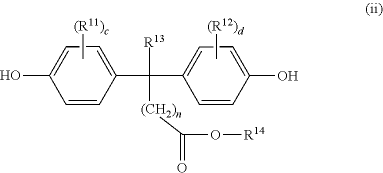
wherein in the formula (ii),
R¹¹ and R¹² each independently represent a halogen atom, or a group selected from the group consisting of: an alkyl group having 1 to 18 carbon atoms; an alkoxy group having 1 to 18 carbon atoms; a cycloalkyl group having 3 to 20 carbon atoms; a cycloalkoxy group having 6 to 20 carbon atoms; an alkenyl group having 2 to 10 carbon atoms; an aryl group having 6 to 14 carbon atoms; an aryloxy group having 6 to 14 carbon atoms; an aralkyl group having 7 to 20 carbon atoms; an aralkyloxy group having 7 to 20 carbon atoms; a nitro group; an aldehyde group; a cyano group; and a carboxyl group,
R¹³ represents a hydrogen atom, or a group selected from the group consisting of: an alkyl group having 1 to 5 carbon atoms; a cycloalkyl group having 3 to 20 carbon atoms; a cycloalkoxy group having 3 to 20 carbon atoms; an alkenyl group having 2 to 10 carbon atoms; and an aryl group having 6 to 14 carbon atoms,
R¹⁴ represents a saturated or unsaturated alicyclic group having 3 to 20 carbon atoms, or a 3- to 20-membered saturated or unsaturated heterocyclic group,
“c” and “d” each independently represent an integer of from 0 to 4, and
“n” represents an integer of from 0 to 20.

11. A method of producing an aromatic polycarbonate-based resin, comprising a step of subjecting a dihydric phenol-based compound and a polycarbonate oligomer to interfacial polycondensation in the presence of a water-insoluble organic solvent and an aqueous solution of an alkali compound, wherein the dihydric phenol-based compound contains a dihydric phenol-based compound (a) represented by the following formula (ii): 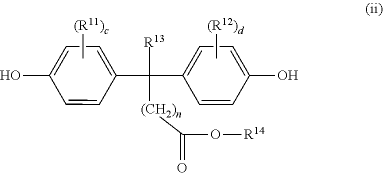
wherein in the formula (ii),
R¹¹ and R¹² each independently represent a halogen atom, or a group selected from the group consisting of: an alkyl group having 1 to 18 carbon atoms; an alkoxy group having 1 to 18 carbon atoms; a cycloalkyl group having 3 to 20 carbon atoms; a cycloalkoxy group having 6 to 20 carbon atoms; an alkenyl group having 2 to 10 carbon atoms; an aryl group having 6 to 14 carbon atoms; an aryloxy group having 6 to 14 carbon atoms; an aralkyl group having 7 to 20 carbon atoms; an aralkyloxy group having 7 to 20 carbon atoms; a nitro group; an aldehyde group; a cyano group; and a carboxyl group,
R¹³ represents a hydrogen atom, or a group selected from the group consisting of: an alkyl group having 1 to 5 carbon atoms; a cycloalkyl group having 3 to 20 carbon atoms; a cycloalkoxy group having 3 to 20 carbon atoms; an alkenyl group having 2 to 10 carbon atoms; and an aryl group having 6 to 14 carbon atoms,
R¹⁴ represents a saturated or unsaturated alicyclic group having 3 to 20 carbon atoms, or a 3- to 20-membered saturated or unsaturated heterocyclic group,
“c” and “d” each independently represent an integer of from 0 to 4, and
“n” represents an integer of from 0 to 20.

12. A polycarbonate-based resin composition, comprising the aromatic polycarbonate-based resin of claim 1.

13. The polycarbonate-based resin composition according to claim 12, wherein the polycarbonate-based resin composition is for use in a scratch-resistant application.

14. A molded article of the polycarbonate-based resin composition of claim 12.

15. The molded article according to claim 14, wherein the molded article is a resin window, a touch panel, an interior supply, an exterior supply, an interior part or an exterior part of a vehicle, a casing, an electric appliance, a building material, or OA equipment.

16. A structural body, comprising an outer surface formed of the polycarbonate-based resin composition of claim 12.

17. A use of the aromatic polycarbonate-based resin of claim 1, or the polycarbonate-based resin composition of claim 1 for a scratch-resistant application.

18. A use of the aromatic polycarbonate-based resin of claim 1, or the polycarbonate-based resin composition of claim 1 for production of a resin window, a touch panel, an interior supply, an exterior supply, an interior part or an exterior part of a vehicle, a casing, an electric appliance, a building material, or OA equipment.

19. A use of a dihydric phenol-based compound represented by the following formula (ii) for production of an aromatic polycarbonate-based resin: 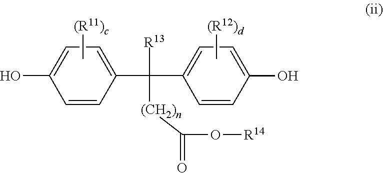
wherein in the formula (ii),
R¹¹ and R¹² each independently represent a halogen atom, or a group selected from the group consisting of: an alkyl group having 1 to 18 carbon atoms; an alkoxy group having 1 to 18 carbon atoms; a cycloalkyl group having 3 to 20 carbon atoms; a cycloalkoxy group having 6 to 20 carbon atoms; an alkenyl group having 2 to 10 carbon atoms; an aryl group having 6 to 14 carbon atoms; an aryloxy group having 6 to 14 carbon atoms; an aralkyl group having 7 to 20 carbon atoms; an aralkyloxy group having 7 to 20 carbon atoms; a nitro group; an aldehyde group; a cyano group; and a carboxyl group,
R¹³ represents a hydrogen atom, or a group selected from the group consisting of: an alkyl group having 1 to 5 carbon atoms; a cycloalkyl group having 3 to 20 carbon atoms; a cycloalkoxy group having 3 to 20 carbon atoms; an alkenyl group having 2 to 10 carbon atoms; and an aryl group having 6 to 14 carbon atoms,
R¹⁴ represents a saturated or unsaturated alicyclic group having 3 to 20 carbon atoms, or a 3- to 20-membered saturated or unsaturated heterocyclic group,
“c” and “d” each independently represent an integer of from 0 to 4, and
“n” represents an integer of from 0 to 20.
